SEQUENCE LISTING (17 sequences)

SEQ ID NO: 1 - DNA, 1650 bp, Bacillus sp.
  CDS (65)..(1567)
  sig_peptide (65)..(128)
  mat_peptide (128)..()
cttgaatcattatttaaagctggttatgatatatgtaagcgttatcattaaaaggaggtatttgatgaaaagatgggtagtagcaatgctggcagtgttatttttatttccttcggtagtagttgcagatggcttgaatggaacgatgatgcagtattatgagtggcatctagagaatgatgggcaacactggaatcggttgcatgatgatgccgaagctttaagtaatgcgggtattacagctatttggatacccccagcctacaaaggaaatagtcaggctgatgttgggtatggtgcatacgacctttatgatttaggggagtttaatcaaaaaggtaccgttcgaacgaaatacgggacaaaggctcagcttgagcgagctatagggtccctaaagtcgaatgatatcaatgtttatggggatgtcgtaatgaatcataaattaggagctgatttcacggaggcagtgcaagctgttcaagtaaatccttcgaaccgttggcaggatatttcaggtgtctacacgattgatgcatggacgggatttgactttccagggcgcaacaatgcctattccgattttaaatggagatggttccattttaatggcgttgactgggatcaacgctatcaagaaaaccatctttttcgctttgcaaatacgaactggaactggcgagtggatgaagagaatggtaattatgactatttattaggatcgaacattgactttagccacccagaggttcaagaggaattaaaggattgggggagctggtttacggatgagctagatttagatgggtatcgattggatgctattaagcatattccattctggtatacgtcagattgggttaggcatcagcgaagtgaagcagaccaagatttatttgtcgtaggggagtattggaaggatgacgtaggtgctctcgaattttatttagatgaaatgaattgggagatgtctctattcgatgttccgctcaattataatttttaccgggcttcaaagcaaggcggaagctatgatatgcgtaatattttacgaggatctttagtagaagcacatccgattcatgcagttacgtttgttgataatcatgatactcagccaggagagtcattagaatcatgggtcgctgattggtttaagccacttgcttatgcgacaatcttgacgcgtgaaggtggttatccaaatgtattttacggtgactactatgggattcctaacgataacatttcagctaagaaggatatgattgatgagttgcttgatgcacgtcaaaattacgcatatggcacacaacatgactattttgatcattgggatatcgttggatggacaagagaaggtacatcctcacgtcctaattcgggtcttgctactattatgtccaatggtcctggaggatcaaaatggatgtacgtaggacagcaacatgcaggacaaacgtggacagatttaactggcaatcacgcggcgtcggttacgattaatggtgatggctggggcgaattctttacaaatggaggatctgtatccgtgtatgtgaaccaataataaaaagccttgagaagggattcctccctaactcaaggctttctttatgtcgtttagctcaacgcttctacgaagcttta

SEQ ID NO: 2 - protein, 501 aa, Bacillus sp.
MKRWVVAMLAVLFLFPSVVVADGLNGTMMQYYEWHLENDGQHWNRLHDDAEALSNAGITAIWIPPAYKGNSQADVGYGAYDLYDLGEFNQKGTVRTKYGTKAQLERAIGSLKSNDINVYGDVVMNHKLGADFTEAVQAVQVNPSNRWQDISGVYTIDAWTGFDFPGRNNAYSDFKWRWFHFNGVDWDQRYQENHLFRFANTNWNWRVDEENGNYDYLLGSNIDFSHPEVQEELKDWGSWFTDELDLDGYRLDAIKHIPFWYTSDWVRHQRSEADQDLFVVGEYWKDDVGALEFYLDEMNWEMSLFDVPLNYNFYRASKQGGSYDMRNILRGSLVEAHPIHAVTFVDNHDTQPGESLESWVADWFKPLAYATILTREGGYPNVFYGDYYGIPNDNISAKKDMIDELLDARQNYAYGTQHDYFDHWDIVGWTREGTSSRPNSGLATIMSNGPGGSKWMYVGQQHAGQTWTDLTGNHAASVTINGDGWGEFFTNGGSVSVYVNQ

SEQ ID NO: 3 - DNA, 1745 bp, Bacillus
  CDS (190)..(1692)
  sig_peptide (190)..(253)
  mat_peptide (253)..()
aactaagtaacatcgattcaggataaaagtatgcgaaacgatgcgcaaaactgcgcaactactagcactcttcagggactaaaccaccttttttccaaaaatgacatcatataaacaaatttgtctaccaatcactatttaaagctgtttatgatatatgtaagcgttatcattaaaaggaggtatttgatgagaagatgggtagtagcaatgttggcagtgttatttttatttccttcggtagtagttgcagatggattgaacggtacgatgatgcagtattatgagtggcatttggaaaacgacgggcagcattggaatcggttgcacgatgatgccgcagctttgagtgatgctggtattacagctatttggattccgccagcctacaaaggtaatagtcaggcggatgttgggtacggtgcatacgatctttatgatttaggagagttcaatcaaaagggtactgttcgaacgaaatacggaactaaggcacagcttgaacgagctattgggtcccttaaatctaatgatatcaatgtatacggagatgtcgtgatgaatcataaaatgggagctgattttacggaggcagtgcaagctgttcaagtaaatccaacgaatcgttggcaggatatttcaggtgcctacacgattgatgcgtggacgggtttcgacttttcagggcgtaacaacgcctattcagattttaagtggagatggttccattttaatggtgttgactgggatcagcgctatcaagaaaatcatattttccgctttgcaaatacgaactggaactggcgagtggatgaagagaacggtaattatgattacctgttaggatcgaatatcgactttagtcatccagaagtacaagatgagttgaaggattggggtagctggtttaccgatgagttagatttggatggttatcgtttagatgctattaaacatattccattctggtatacatctgattgggttcggcatcagcgcaacgaagcagatcaagatttatttgtcgtaggggaatattggaaggatgacgtaggtgctctcgaattttatttagatgaaatgaattgggagatgtctctattcgatgttccacttaattataatttttaccgggcttcacaacaaggtggaagctatgatatgcgtaatattttacgaggatctttagtagaagcgcatccgatgcatgcagttacgtttgttgataatcatgatactcagccaggggagtcattagagtcatgggttgctgattggtttaagccacttgcttatgcgacaattttgacgcgtgaaggtggttatccaaatgtattttacggtgattactatgggattcctaacgataacatttcagctaaaaaagatatgattgatgagctgcttgatgcacgtcaaaattacgcatatggcacgcagcatgactattttgatcattgggatgttgtaggatggactagggaaggatcttcctccagacctaattcaggccttgcgactattatgtcgaatggacctggtggttccaagtggatgtatgtaggacgtcagaatgcaggacaaacatggacagatttaactggtaataacggagcgtccgttacaattaatggcgatggatggggcgaattctttacgaatggaggatctgtatccgtgtacgtgaaccaataacaaaaagccttgagaagggattcctccctaactcaaggctttctttatgt

SEQ ID NO: 4 - protein, 501 aa, Bacillus
MRRWVVAMLAVLFLFPSVVVADGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 5 - DNA, 1920 bp, Bacillus licheniformis
  CDS (421)..(1872)
cggaagattggaagtacaaaaataagcaaaagattgtcaatcatgtcatgagccatgcgggagacggaaaaatcgtcttaatgcacgatatttatgcaacgttcgcagatgctgctgaagagattattaaaaagctgaaagcaaaaggctatcaattggtaactgtatctcagcttgaagaagtgaagaagcagagaggctattgaataaatgagtagaagcgccatatcggcgcttttcttttggaagaaaatatagggaaaatggtacttgttaaaaattcggaatatttatacaacatcatatgtttcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaggcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatttcaggagaacacctaattaaagcctggacacattttcattttccggggcgcggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggtttccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaacggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttatttt

SEQ ID NO: 6 - protein, 483 aa, Bacillus licheniformis [sequence omitted]

SEQ ID NO: 7 - DNA, 1776 bp, Bacillus sp.
  CDS (145)..(1692)
  sig_peptide (145)..(238)
  mat_peptide (238)..()
atataaatttgaaatgaacacctatgaaaatatggtagcgattgcgcgacgagaaaaaacttgggagttaggaagtgatattaaaggattttttttgacttgttgtgaaaacgcttgcataaattgaaggagagggtgctttttatgaaacttcataaccgtataattagcgtactattaacactattgttagctgtagctgttttgtttccatatatgacggaaccagcacaagcccatcataatgggacgaatgggaccatgatgcagtattttgaatggcatttgccaaatgacgggaaccactggaacaggttacgagatgacgcagctaacttaaagagtaaagggattaccgctgtttggattcctcctgcatggaaggggacttcgcaaaatgatgttgggtatggtgcctatgatttgtacgatcttggtgagtttaaccaaaagggaaccgtccgtacaaaatatggcacaaggagtcagttgcaaggtgccgtgacatctttgaaaaataacgggattcaagtttatggggatgtcgtgatgaatcataaaggtggagcagacgggacagagatggtaaatgcggtggaagtgaaccgaagcaaccgaaaccaagaaatatcaggtgaatacaccattgaagcatggacgaaatttgatttccctggaagaggaaatacccattccaactttaaatggcgctggtatcattttgatgggacagattgggatcagtcacgtcagcttcagaacaaaatatataaattcagaggtaccggaaaggcatgggactgggaagtagatatagagaacggcaactatgattaccttatgtatgcagacattgatatggatcatccagaagtaatcaatgaacttagaaattggggagtttggtatacaaatacacttaatctagatggatttagaatcgatgctgtgaaacatattaaatacagctatacgagagattggctaacacatgtgcgtaacaccacaggtaaaccaatgtttgcagttgcagaattttggaaaaatgaccttgctgcaatcgaaaactatttaaataaaacaagttggaatcactccgtgttcgatgttcctcttcattataatttgtacaatgcatctaatagtggtggctattttgatatgagaaatattttaaatggttctgtcgtacaaaaacaccctatacatgcagtcacatttgttgataaccatgactctcagccaggagaagcattggaatcctttgttcaatcgtggttcaaaccactggcatatgcattgattctgacaagggagcaaggttacccttccgtattttacggtgattactacggtataccaactcatggtgttccttcgatgaaatctaaaattgatccacttctgcaggcacgtcaaacgtatgcctacggaacccaacatgattattttgatcatcatgatattatcggctggacgagagaaggggacagctcccacccaaattcaggacttgcaactattatgtccgatgggccagggggtaataaatggatgtatgtcgggaaacataaagctggccaagtatggagagatatcaccggaaataggtctggtaccgtcaccattaatgcagatggttgggggaatttcactgtaaacggaggggcagtttcggtttgggtgaagcaataaataaggaacaagaggcgaaaattactttcctacatgcagagctttccgatcactcatacacccaatataaattggaagctt

SEQ ID NO: 8 - protein, 516 aa, Bacillus sp.
MKLHNRIISVLLTLLLAVAVLFPYMTEPAQAHHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 9 - DNA, 40 bp, Artificial sequence
  Primer DAX-8N
gctgcggccgctgcagatggmttgaayggwacgatgatgc

SEQ ID NO: 10 - DNA, 43 bp, Artificial sequence
  Primer DAX-8C
ggccgtcgacttattggttcacrtacacggatacagatcctcc

SEQ ID NO: 11 - DNA, 32 bp, Artificial sequence
  Primer Amrk752-E84Q
ctaaggcacagcttcaacgagctattgggtcc

SEQ ID NO: 12 - DNA, 35 bp, Artificial sequence
  Primer Amrk752-N96D
ccttaaatctaatgatatcgatgtatacggagatg

SEQ ID NO: 13 - DNA, 34 bp, Artificial sequence
  Primer Amrk752-A315S
ttataatttttaccggtcttcacaacaaggtgga

SEQ ID NO: 14 - DNA, 32 bp, Artificial sequence
  Primer Amrk752-A445V
gtaggacgtcagaatgtaggacaaacatggac

SEQ ID NO: 15 - DNA, 35 bp, Artificial sequence
  Primer Amrk752-G464N
ccgttacaattaataacgatggatggggcgaattc

SEQ ID NO: 16 - DNA, 34 bp, Artificial sequence
  Primer Amrk230-N121D
gcaagctgttcaagtagatccaacgaatcgttgg

SEQ ID NO: 17 - DNA, 34 bp, Artificial sequence
  Primer Amrk230-N390H
gcttgatgcacgtcaagattacgcatatggcacg

(19) United States
(12) Patent Application Publication
(10) Pub. No.: US 2004/0091994 A1
(43) Pub. Date: May 13, 2004

(54) Alpha-amylase variant with altered properties

(76) Inventor: Carsten Andersen, Vaerlose (DK)

Correspondence Address: NOVOZYMES NORTH AMERICA, INC., 500 FIFTH AVENUE, SUITE 1600, NEW YORK, NY, 10110, US

(21) Appl. No.: 10/399,161

(22) PCT Filed: Oct. 12, 2001
(86) PCT No.: PCT/DK01/00668

(30) Foreign Application Priority Data
Oct. 13, 2000 (PA) 2000 01533
Oct. 2, 2001 (PA) 2001-01442

(51) Int. Cl. C12N 9/28; C11D 3/386; C07H 21/04; C11D 7/42; C12P 21/02; C12N 1/21; C12N 15/74

(52) U.S. Cl. 435/202; 510/320; 435/69.1; 435/320.1; 435/252.3; 536/23.2; 510/220

(57) ABSTRACT

The present invention relates to variants of parent alpha-amylases, which variant has alpha-amylase activity and exhibits an alteration in at least one of the following properties relative to said parent alpha-amylase: substrate specificity, substrate binding, substrate cleavage pattern, thermal stability, pH/activity profile, pH/stability profile, stability towards oxidation, specific activity, and altered pI, in particular higher pI.

### FIELD OF THE INVENTION

The present invention relates to variants (mutants) of parent alpha-amylases, in particular of Bacillus origin, which variant has alpha-amylase activity and exhibits an alteration in at least one of the following properties relative to said parent alpha-amylase: substrate specificity, substrate binding, substrate cleavage pattern, thermal stability, pH/activity profile, pH/stability profile, stability towards oxidation, specific activity, and pI, in particular higher pI.

### BACKGROUND OF THE INVENTION

Alpha-Amylases (alpha-1,4glucan-4-glucanohydrolases, E.C. 3.2.1.1) constitute a group of enzymes, which catalyze hydrolysis of starch and other linear and branched 1,4-glucosidic oligo- and polysaccharides.

The object of the invention is to provide an improved alpha-amylase, in particular suitable for detergent use.

### BRIEF DISCLOSURE OF THE INVENTION

The object of the present invention is to provide an alpha-amylases which variants in comparison to the corresponding parent alpha-amylase, i.e., un-mutated alpha-amylase, has alpha-amylase activity and exhibits an alteration in at least one of the following properties relative to said parent alpha-amylase: substrate specificity, substrate binding, substrate cleavage pattern, thermal stability, pH/activity profile, pH/stability profile, stability towards oxidation, Ca²⁺ dependency, specific activity, and pI.

### Nomenclature

In the present description and claims, the conventional one-letter and three-letter codes for amino acid residues are used. For ease of reference, alpha-amylase variants of the invention are described by use of the following nomenclature:

Original amino acid(s): position(s): substituted amino acid(s)

According to this nomenclature, for instance the substitution of alanine for asparagine in position 30 is shown as:

Ala30Asn or A30N

a deletion of alanine in the same position is shown as:

Ala30* or A30*

and insertion of an additional amino acid residue, such as lysine, is shown as:

Ala30AlaLys or A30AK

A deletion of a consecutive stretch of amino acid residues, such as amino acid residues 30-33, is indicated as (30-33)* or Δ(A30-N33).

Where a specific alpha-amylase contains a “deletion” in comparison with other alpha-amylases and an insertion is made in such a position this is indicated as:

*36Asp or *36D

for insertion of an aspartic acid in position 36.

Multiple mutations are separated by plus signs, i.e.:

Ala30Asp+Glu34Ser or A30N+E34S

representing mutations in positions 30 and 34 substituting alanine and glutamic acid for asparagine and serine, respectively.

When one or more alternative amino acid residues may be inserted in a given position it is indicated as

A30N,E or

A30N or A30E

Furthermore, when a position suitable for modification is identified herein without any specific modification being suggested, it is to be understood that any amino acid residue may be substituted for the amino acid residue present in the position. Thus, for instance, when a modification of an alanine in position 30 is mentioned, but not specified, it is to be understood that the alanine may be deleted or substituted for any other amino acid, i.e., any one of:

R,N,D,A,C,Q,E,G,H,I,L,K,M,F,P,S,T,W,Y,V.

Further, “A30X” means any one of the following substitutions:

A30R, A30N, A30D, A30C, A30Q, A30E, A30G, A30H, A30I, A30L, A30K, A30M, A30F, A30P, A30S, A30T, A30W, A30Y, or A30 V; or in short: A30R,N,D,C,Q,E,G,H,I,L,K,M,F,P,S,T,W,Y,V.

### BRIEF DESCRIPTION OF THE DRAWINGS

FIG. 1 is an alignment of the amino acid sequences of five parent alpha-amylases. The numbers on the extreme left designate the respective amino acid sequences as follows:

1: SEQ ID NO: 6 (Bacillus licheniformis alpha-amylase)

2: SEQ ID NO: 8 (KSM-AP1378 alpha-amylase)

3: SEQ ID NO: 2 (KSM-K36 alpha-amylase)

4: SEQ ID NO: 4 (KSM-K38 alpha-amylase)

### DETAILED DISCLOSURE OF THE INVENTION

The object of the present invention is to provide an alpha-amylases, in particular of Bacillus origin, which variants has alpha-amylase activity and exhibits an alteration in at least one of the following properties relative to said parent alpha-amylase: substrate specificity, substrate binding, substrate cleavage pattern, thermal stability, pH/activity profile, pH/stability profile, stability towards oxidation, specific activity, and altered pI, in particular higher pI.

### Parent Alpha-Amylases

Contemplated alpha-amylases include the alpha-amylases shown in SEQ ID NO: 2 or SEQ ID NO: 4 of Bacillus origin and alpha-amylase having at least 70% identity thereto. The SEQ ID NO: 1 shows the DNA sequence encoding KSM-K36 and SEQ ID NO: 3 show the DNA sequence encoding KSM-K38. These two alpha-amylases are disclosed in EP 1,022,334 (hereby incorporated by reference).

The KSM-K38 alpha-amylase has about 67% identity with the KSM-AP1378 alpha-amylase disclosed in WO 97/00324); 64% identity with the #707 alpha-amylase derived from Bacillus sp.#707 disclosed by Tsukamoto et al., Biochemical and Riophysical Research Communications, 151 (1988), pp. 25-31; and about 63% identity with the Bacilius licheniformis alpha-amylase described in EP 0252666 (ATCC 27811).

Other alpha-amylases within the scope of the present invention include alpha-amylases i) which displays at least 70%, such as at least 75%, or at least 80%, at least 85%, at least 90%, at least 95%, at least 97%, at least 99% homology with at least one of said amino acid sequences shown in SEQ ID NOS: 2 or 4, and/or ii) is encoded by a DNA sequence which hybridizes to the DNA sequences encoding the above-specified alpha-amylases which are apparent from SEQ ID NOS: 1 or 3.

In connection with property i), the homology may be determined as the degree of identity between the two sequences indicating a derivation of the first sequence from the second. The homology may suitably be determined by means of computer programs known in the art such as GAP provided in the GCG program package (described above). Thus, Gap GCGv8 may be used with the following default parameters: GAP creation penalty of 5.0 and GAP extension penalty of 0.3, default scoring matrix. GAP uses the method of Needleman/Wunsch/Sellers to make alignments.

Alternatively, the software Clustal X obtainable from EMBL (ftp-embl-heidelberg.de) may be used for multiple alignments with a gap creation penalty of 30, a gap extension penalty of 1 without gap penalty.

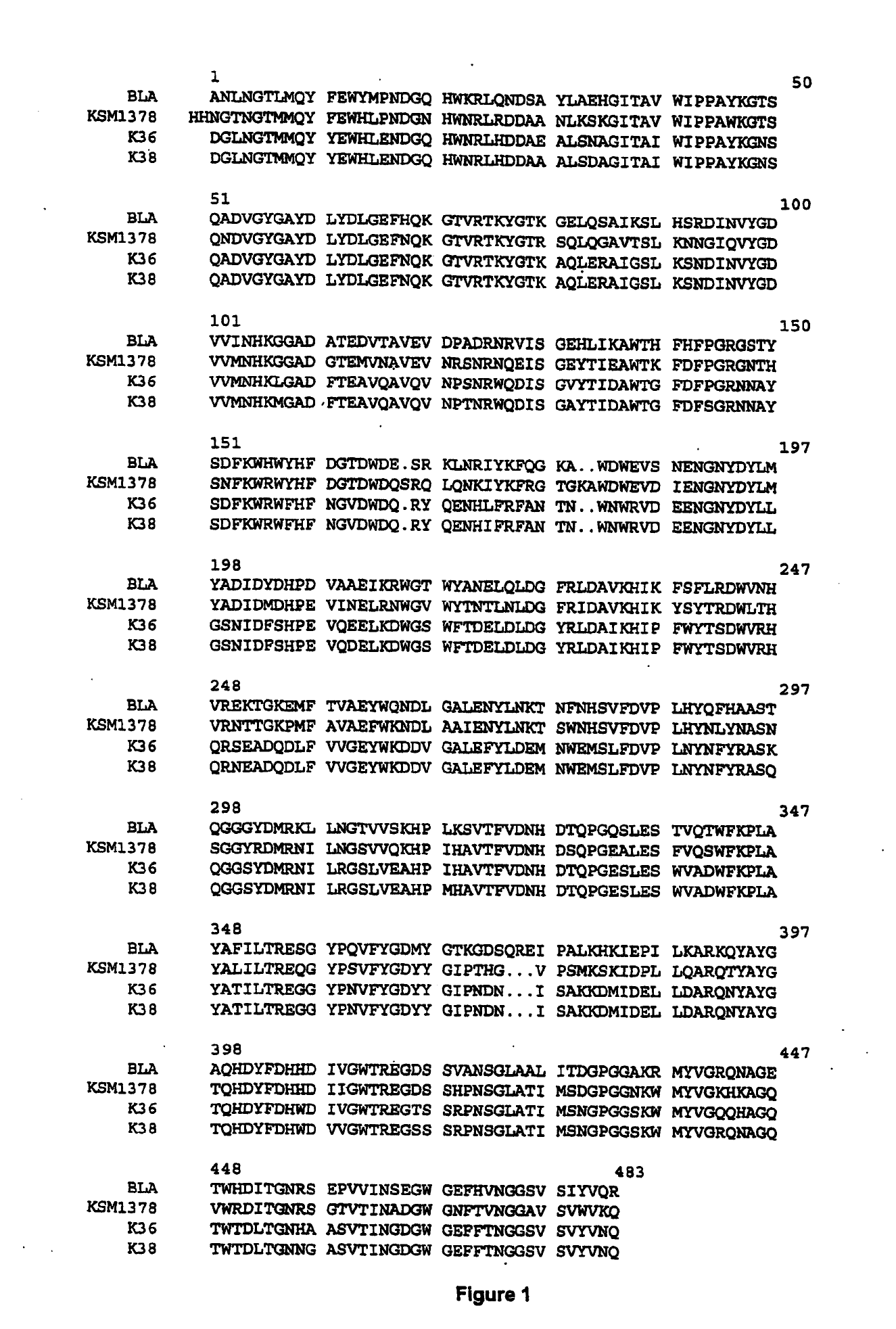

A structural alignment between the KSM-K36 or KSM-K38 alpha-amylases (SEQ ID NO: 2 and 4) and other alpha-amylase may be used to identify equivalent/corresponding positions in other alpha-amylases. One method of obtaining said structural alignment is to use the Pile Up programme from the GCG package using default values of gap penalties, i.e., a gap creation penalty of 3.0 and gap extension penalty of 0.1. Other structural alignment methods include the hydrophobic cluster analysis (Gaboriaud et al., (1987), FEBS LETTERS 224, pp. 149-155) and reverse threading (Huber, T; Torda, AE, PROTEIN SCIENCE Vol. 7, No. 1 pp. 142-149 (1998). An alignment of the KSM-K36, KSM-K38, KSM-AP1378 and the Bacillus licheniformis alpha-amylase is shown in FIG. 1.

### Hybridisation

The oligonucleotide probe used in the characterisation of the KSM-K36 or KSM-K38 alpha-amylases in accordance with property ii) above may suitably be prepared on the basis of the full or partial nucleotide or amino acid sequence of the alpha-amylase in question.

Suitable conditions for testing hybridisation involve pre-soaking in 5×SSC and prehybridizing for 1 hour at ˜40° C. in a solution of 20% formamide, 5×Denhardt's solution, 50 mM sodium phosphate, pH 6.8, and 50 mg of denatured sonicated calf thymus DNA, followed by hybridisation in the same solution supplemented with 100 mM ATP for 18 hours at 40° C., followed by three times washing of the filter in 2×SSC, 0.2% SDS at 40° C. for 30 minutes (low stringency), preferred at 50° C. (medium stringency), more preferably at 65° C. (high stringency), even more preferably at 75° C. (very high stringency). More details about the hybridisation method can be found in Sambrook et al., Molecular_Cloning: A Laboratory Manual, 2nd Ed., Cold Spring Harbor, 1989.

In the present context, “derived from” is intended not only to indicate an alpha-amylase produced or producible by a strain of the organism in question, but also an alpha-amylase encoded by a DNA sequence isolated from such strain and produced in a host organism transformed with said DNA sequence. Finally, the term is intended to indicate an alpha-amylase, which is encoded by a DNA sequence of synthetic and/or cDNA origin and which has the identifying characteristics of the alpha-amylase in question. The term is also intended to indicate that the parent alpha-amylase may be a variant of a naturally occurring alpha-amylase, i.e., a variant, which is the result of a modification (insertion, substitution, deletion) of one or more amino acid residues of the naturally occurring alpha-amylase.

### Altered Properties

The following discusses the relationship between mutations, which are present in variants of the invention, and desirable alterations in properties (relative to those a parent KSM-K36 or KSM-K38 alpha-amylases), which may result therefrom.

As mentioned above the invention relates to alpha-amylase variants with altered properties.

In an aspect the invention relates to variant with altered properties as mentioned above.

In the first aspect a variant of a parent KSM-K36 or KSM-K38 alpha-amylase, comprising an alteration at one or more positions (using SEQ ID NO: 2 or 4 for the amino acid numbering) selected from the group of:

2,9,14,15,16,26,27,48,49,51,52,53,54,58,73,88,94,96,103,104,107,108,111,114,128,130,133, 138,140,142,144,148,149,156,161,165,166,168,171,173,174,178,179,180,181,183,184,187, 188,190,194,197,198,199,200,201,202,203,204,205,207,209,210,211,212,214,221,222,224, 228,230,233,234,237,239,241,242,252,253,254,255,260,264,265,267,275,276,277,280,281, 286,290,293,301,305,314,315,318,329,333,340,341,356,375,376,377,380,383,384,386,389, 399,403,404,405,406,427,441,444,453,454,472,479,480

wherein

(a) the alteration(s) are independently

(i) an insertion of an amino acid downstream of the amino acid which occupies the position,

(ii) a deletion of the amino acid which occupies the position, or

(iii) a substitution of the amino acid which occupies the position with a different amino acid,

(b) the variant has alpha-amylase activity and (c) each position corresponds to a position of the amino acid sequence of the parent alpha-amylase having the amino add sequence of the KSM-K36 alpha-amylases shown in SEQ ID NO: 2.

In the KSM-38 alpha-amylase the target positions are:

2,9,14,15,16,26,27,48,49,51,52,53,54,58,73,88,94,96,103,104,107,108,111,114,128,130,133, 138,140,142,144,148,149,156,161,165,166,168,171,173,174,178,179,180,181,183,184,187, 188,190,194,197,198,199,200,201,202,203,204,205,207,209,210,211,212,214,221,222,224, 228,230,233,234,237,239,241,242,252,253,254,255,260,264,265,267,275,276,277,280,281, 286,290,293,301,305,314,315,318,329,333,340,341,356,375,376,377,380,383,384,386,389, 399,403,404,405,406,427,441,444,453,454,472,479,480.

In a preferred embodiment of the invention the variant comprise one or more of the following substitutions (using SEQ ID NO: 2 for the numbering):

G2P,A; M91,L,F; H14Y; L15M,I,F,T; E16P; H26Y,Q,R,N; D27N,S,T; G48A,V,S,T; N49X; Q51X; A52X; D53E,Q,R; V54X; A58V,L,I,F; V73L,I,F; E84Q; G88X; D94X; N96Q; M103I,L,F; N104D; M/L107G,A,V,T,S; G108A; F111G,A,V,I,L,T; A114D,I,L,M,V,R; T125S; D128T,E; S130T,C; Y133F,H; W138F,Y; G140H,R,K,D,N; D142H,R,K,N; S144P; N148S; A149I; R156H,K,D,N; N161X; W165R; D166E; R168P; E171L,I,F; H173R,K,L; I173L; L174I,F; A178N,Q,R,K,H; N179G,A,T,S; T180N,Q,R,K,H; N181X; N183X; W184R,K; D187N,S,T; E188P,T,I,S; N190F; D194X; L197X; G198X; S199X; N200X; I201L,M,F,Y; D202X; F203L,I,F,M; S204X;H205X; E207Y,R; Q209V,L,I,F,M; E210X; E211Q; L212I,F; D214N,R,K,H; D221N; E222Q,T; D224N,Q; Y228F; L230I,F; I233A,V,L,F; K234N,Q; P237X; W239X; T241L,I,F,M; S242P,R; A252T; D253G,A,V,N; Q254K; D255N,Q,E,P; G260A; K264Q,S,T; D265N,Y; V267L,I,F,M; D275N,T; E276K; M277T,I,L,F; E280N,T,Q,S; M281H,I,L,F; V286X, preferably V286Y,L,I,F; Y290X; Y293H,F; S301 G,A,D,K,E,R; R305A,K,Q,E,H,D,N; E314K,Q,R,S,T,H,N; A315K,R,S; I318L,M,F; T329S; E333Q; A340R,K,N,D,Q,E; D341P,T,S,Q,N; G356Q,E,S,T,A; S375P; A376S; K377L,I,F,M; M380I,L,F; E383P,Q; L384I,F; D386N,Q,R,K,I,L; Q389K,R; Y399A,D,H; W403X; D404N; I405L,F; V406I,L,F,A,D; N427X; H441 K,N,D,Q,E; R442Q; Q444E,K,R; A445V; Q448A; H453R,K,Q,N; A454S,T,P; G472R, N479Q,K,R; Q480K,R.

In another preferred embodiment of the invention the variant comprise one or more of the following substitutions (using SEQ ID NO: 4 for the numbering):

G2P,A; M9I,L,F; H14Y; L15M,I,F,T; E16P; H26Y,Q,R,N; D27N,S,T; G48A,V,S,T; N49X; Q51X; A52X; D53E,Q,R; V54X; A58V,L,I,F; V73L,I,F; E84Q; G88X; D94X; N96Q; M103I,L,F; N104D; M/L107G,A,V,T,S,I,L,F; G108A; F111G,A,V,I,L,T; A114D,I,L,M,V,R; T125S; D128T,E; S130T,C; Y133F,H; W138F,Y; G140H,R,K,D,N; D142H,R,K,N; S144P; N148S; A149I; R156H,K,D,N; N161X; W165R; D166E; R168P; E171L,I,F; H173R,K,L; I173L; I174L,F; A178N,Q,R,K,H; N179G,A,T,S; T180N,Q,R,K,H; N181X; N183X; W184R,K; D187N,S,T; E188P,T,I,S; N190F;D194X; L197X; G198X; S199X; N200X; I201L,M,F,Y; D202X;. F203L,I,F,M; S204X; H205X; E207Y,R; Q209V,L,I,F,M; D210X; D210E; E211Q; L212I,F; D214N,R,K,H; D221N; E222Q,T; D224N,Q; Y228F; L230I,F; I233A,V,L,F; K234N,Q; P237X; W239X; T241L,I,F,M; S242P,R; A252T; D253G,A,V,N; Q254K; D255N,Q,E,P; G260A; K264Q,S,T; D265N,Y; V267L,I,F,M; D275N,T; E276K; M277T,I,L,F; E280N,T,Q,S; M281H,I,L,F; V286X, preferably V286Y,L,I,F; Y290X; Y293H,F; S301G,A,D,K,E,R; R305A,K,Q,E,H,D,N; E314K,Q,R,S,T,H,N; A315K,R,S; M318L,I,F; T329S; E333,Q; A340R,K,N,D,Q,E; D341P,T,S,Q,N; G356Q,E,S,T,A; S375P; A376S; K377L,I,F,M; M380I,L,F; E383P,Q; L384I,F; D386N,Q,R,K,I,L; Q389K,R; Y399A,D,H; W403X; D404N; V405L,F,I; V406I,L,F,A,D; N427X; N441K,D,Q,E,H; R442Q; Q444E,K,R; A445V; Q448A; N453R,K,Q,H; G454A,S,T,P; G472R, N479Q,K,R; Q480K,R.

Within the scope of the invention are variants of other parent alpha-amylases (as defined herein) with one or more corresponding mutations.

In an embodiment of the invention a variant of the invention may comprise the following combination of substitutions:

N49I+L/M107A;

N49L+L/M107A;

G48A+N49I+L/M 107A;

G48A+N49L+L/M107A;

E188S,T,P+N190F+I201F+K264S;

G48A+N49I+L/M107A+E188S,T,P+N190F+I201 F+K264S;

N190F+I201F;

N190F+K264S;

I201F+K264S;

G140H+D142H+R156H,Y(+S144P);

G140K+D142D+R156H,Y(+S144P);

L197M+G198Y+S199A;

L15T+E188S+Q209V+A376S+G472R;

G48A+N49I+L/M107A+G140H+D142H+R156H,Y+E188P+N190F+I201F+K264S(+144P); N49T+L/M107A+G140H+D142H+R156H,Y+E188P+N190F+I201F+K264S(144P); “/” before number indicate that KSM-K36 and KSM-K38 has different amino acid on the actual position. For instance L/M107A means that in KSM-K36 the mutation is L107A and in KSM-K38 the substitution is M107A.

### Altered pI

Important positions and mutations with respect to achieving altered pI, in particular a higher pI, in particular at high pH (ie., pH 8-10.5) include any of the positions and mutations listed in the in “Altered Properties” section. It should be noted that when the alpha-amylase of the invention is for detergent use a high pI is desirable—for instance a pI in the range from 7-10.

### Stability

Important positions and mutations with respect to achieving altered stability, in particular improved stability (i.e., higher or lower) at especially high pH (i.e., pH 8-10.5) include any of the positions and mutations listed in the in “Altered Properties” section.

### Oxidation Stability

Variants of the invention may have altered oxidation stability, in particular higher oxidation stability, in comparison to the parent alpha-amylase.

In an embodiment such an alpha-amylase variant has one or more of Methionine amino acid residues exchanged with any amino acid residue except for Cys and Met. Thus, according to the invention the amino acid residues to replace the Methionine amino acid residue are the following: Ala, Arg, Asn, Asp, Gln, Glu, Gly, His, Ile, Leu, Lys, Phe, Pro, Ser, Thr, Trp, Tyr, and Val.

A preferred embodiment of the alpha-amylase of the invention is characterized by the fact that one or more of the Methionine amino acid residues is (are) exchanged with a Phe, Leu, Thr, Ala, Gly, Ser, Ile, or Val amino acid residue, preferably a Leu, Ile, Phe amino acid residue. In this embodiment a very satisfactory activity level and stability in the presence of oxidizing agents is obtained. Specifically this means that one or more of the Methionines in the following position may be replaced or deleted using any suitable technique known in the art, including especially site directed mutagenesis and gene shuffling. Target Methionine positions, using the SEQ ID NO: 2 numbering (KSM-K36), are one or more of M7, M8, M103, N277, M281, M304, M383, M428, M438.

Target Methionine positions, using the SEQ ID NO: 4 numbering (KSM-K38), are one or more of M7, M8, M103, M107, M197, M277, M281, M304, M318, M383, M428, M438.

In a preferred embodiment of the mutant alpha-amylase of the invention is characterized by the fact that the Methionine amino acid residue at position M107 and/or M277 and/or M281, and/or M318 and/or M383 and/or M428 is(are) exchanged with any of amino acid residue expect for Cys and Met, preferably with a Phe, Leu, Thr, Ala, Gly, Ser, Ile, or Asp. Also other parent alpha-amylases, as defined above, may have one or more Methionines substituted or deleted in particular in corresponding positions.

### Specific Activity

Important positions and mutations with respect to obtaining variants exhibiting altered specific activity, in particular increased or decreased specific activity, especially at temperatures from 10-60° C., preferably 20-50° C., especially 30-40° C., include any of the below positions and substututions. The amino acid residues of particular importance are those involved in substrate binding. Primary target positions, using the SEQ ID NO: 2 numbering (KSM-K36), are one or more of G48, N49, Q51, A52, D53, V54, L107, G108, W165, D166, L197, G198, S199, K234, K264.

Primary target positions, using the SEQ ID NO: 4 numbering (KSM-K38), are one or more of G48, N49, Q51, A52, D53, V54, M107, G108, W165, D166, L197, G198, S199, K234, K264.

Preferred specific mutations/substitutions are the ones listed above in the section “Altered Properties” for the positions in question.

### Altered pH Profile

Important positions and mutations with respect to obtaining variants with altered pH profile, in particular improved activity at especially low pH (i.e., pH 4-6) include mutations of amino residues located close to the active site residues, i.e., D229, E261, D328. Primary target positions, using the SEQ ID NO: 2 numbering (KSM-K36), are one or more of N104, E333

Primary target positions, using the SEQ ID NO: 4 numbering (KSM-K38), are one or more of N104, E333.

Preferred specific mutations/substitutions are the ones listed above in the section “Altered properties” for the positions in question.

### Altered Alpha-Amylase Activity

A variant of the invention may have altered alpha-amylase activity, in particular increased alpha-amylase activity, in comparison to the parent alpha-amylase using the Phadebas® assay described below in the “Materials & Methods” section.

In a preferred embodiment of the invention an alpha-amylase substituted in a position corresponding to position 286 using the SEQ ID NO: 2 for the numbering has increased alpha-amylase activity. Preferred substitutions are V286Y,L,I,F.

In Bacillus sp. (SEQ ID NO: 2) the following substitution are result in increased activity: V286X (i.e., V286A,R,N,D,C,Q,E,G,H,I,L,K,M,F,P,S,T,W,Y), preferred are V286Y,L,I,F.

In Bacillus sp. (SEQ ID NO: 4) the following substitution are result in increased activity: A286X (i.e., V286R,N,D,C,Q,E,G,H,I,L,K,M,F,P,S,T,W,Y,V), preferred are A286Y,L,I,F.

### Other Mutations

Other preferred mutations to increase the stability of a particular protein include substitutions to a similar amino acid having a larger side chain, in order to fill out internal holds in the globular structure. Examples of these include glycine to alanine, alanine to valine, valine to isoleucine or leucine, alanine to serine, serine to threonine, asparagine to glutamine, asparatate to glutamate, phenyl to tyrosine or tryptophane, tyrosine to tryptophane, asparagine or asparatate to histidine, histidine to tyrosine and lysine to arginine substitutions, but are not limited to these examples only. Preferred mutations are Q84E, N96D, N121D, N393H, N444H.

Examples of larger mutations in SEQ 2 include: A315S/V and V101I. Examples of larger mutations in SEQ 4 include: V101I, A132V, D210E, A315SN, V408I, S416T and A447V. Also other parent alpha-amylases, as defined above, may have one or more amino acid substituted into a larger amino acid, in particular in corresponding positions.

Other preferred mutations include substitutions of glycine residues to decrease the flexibility of the protein backbone. Examples of glycine substitutions in SEQ 2 and 4 includes: 19, 36, 48, 55, 57, 65, 71, 82, 88, 99, 108, 131, 140, 145, 162, 191, 198, 216, 227, 260, 268, 299, 300, 310, 332, 356, 357, 364, 368, 397, 410, 415, 423, 431, 433, 432, 441, 447, 454, 457, 464, 466, 468, 474, 475 and in particular G464A/S/N. Also other parent alpha-amylases, as defined above, may have one or more glycines substituted or deleted in particular in corresponding positions.

Other preferred mutations include introduction of proline residues in positions where possible with respect to limitations in the dihedral angles of the protein backbone and in the secondary structure. Examples of substitutions into proline in SEQ 2 include: W13, E16, Q51, L61, A109, G131, W182, D187, I233, I307, S334, W338, D341, W342, A379, S417.

Examples of substitutions into proline in SEQ 4 include: W13, E16, Q51, L61, A109, G131, S144, W182, D187, I233, I307, S334, W338, D341, W342, A379, S417. Also other parent alpha-amylases, as defined above, may be stabilised by introduction of a proline, in particular in corresponding positions.

Important positions and mutations with respect to obtaining variants with improved stability at low pH are Aspargine substitutions. Preferred mutations include substitution or deletion of one or more Aspargine (Asn). Target Aspargines in SEQ ID NO: 2 (KSM-36) are N4, N17, N23, N34, N49, N68, N93, N96, N104, N121, N124, N147, N148, N161, N172, N179, N181, N183, N190, N192, N200, N278, N289, N291, N306, N326, N360, N371, N373, N393, N421, N430, N455, N463, N473, N482, which may be substituted with any other amino acid, or deleted, in particular N190F.

Target Aspargines in SEQ ID NO: 4 (KSM-38) are N17, N23, N49, N68, N93, N96, N104, N121, N124, N147, N148, N161, N172, N179, N181, N183, N190, N192, N200, N250, N278, N289, N291, N306, N326, N360, N371, N373, N393, 421, N430, N444, N455, N456, N463, N473, N482, which may be substituted with any other amino acid, or deleted, in particular N190F.

Also other parent alpha-amylases, as defined above, may have one or more Aspargines substituted or deleted in particular in corresponding positions.

### Methods for Preparing Alpha-Amylase Variants of the Invention

Several methods for introducing mutations into genes are known in the art. After a brief discussion of the cloning of alpha-amylase-encoding DNA sequences, methods for generating mutations at specific sites within the alpha-amylase-encoding sequence will be discussed.

### Cloning a DNA Sequence Encoding an Alpha-Amylase

The DNA sequence encoding a parent alpha-amylase may be isolated from any cell or microorganism producing the alpha-amylase in question, using various methods well known in the art. First, a genomic DNA and/or cDNA library should be constructed using chromosomal DNA or messenger RNA from the organism that produces the alpha-amylase to be studied. Then, if the amino acid sequence of the alpha-amylase is known, homologous, labeled oligonucleotide probes may be synthesized and used to identify alpha-amylase-encoding clones from a genomic library prepared from the organism in question. Alternatively, a labeled oligonucleotide probe containing sequences homologous to a known alpha-amylase gene could be used as a probe to identify alpha-amylase-encoding clones, using hybridization and washing conditions of lower stringency.

Yet another method for identifying alpha-amylase-encoding clones would involve inserting fragments of genomic DNA into an expression vector, such as a plasmid, transforming alpha-amylase-negative bacteria with the resulting genomic DNA library, and then plating the transformed bacteria onto agar containing a substrate for alpha-amylase, thereby allowing clones expressing the alpha-amylase to be identified.

Alternatively, the DNA sequence encoding the enzyme may be prepared synthetically by established standard methods, e.g., the phosphoroamidite method described by S. L. Beaucage and M. H. Caruthers, Tetrahedron Letters 22, 1981, pp. 1859-1869, or the method described by Matthes et al., The EMBO J. 3, 1984, pp. 801-805. In the phosphoroamidite method, oligonucleotides are synthesized, e.g., in an automatic DNA synthesizer, purified, annealed, ligated and cloned in appropriate vectors.

Finally, the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in U.S. Pat. No. 4,683,202 or R. K. Saiki et al., Science 239, 1988, pp. 487-491.

### Site-Directed Mutagenesis

Once an alpha-amylase-encoding DNA sequence has been isolated, and desirable sites for mutation identified, mutations may be introduced using synthetic oligonucleotides. These oligonucleotides contain nucleotide sequ nces flanking the desired mutation sites; mutant nucleotides are inserted during oligonucleotide synthesis. In a specific method, a single-stranded gap of DNA, bridging the alpha-amylase-encoding sequence, is created in a vector carrying the alpha-amylase gene. Then the synthetic nucleotide, bearing the desired mutation, is annealed to a homologous portion of the single-stranded DNA. The remaining gap is then filled in with DNA polymerase I (Klenow fragment) and the construct is ligated using T4 ligase. A specific example of this method is described in Morinaga et al., (1984, Biotechnology 2:646-639). U.S. Pat. No. 4,760,025 discloses the introduction of oligonucleotides encoding multiple mutations by performing minor alterations of the cassette. However, an even greater variety of mutations can be introduced at any one time by the Morinaga method, because a multitude of oligonucleotides, of various lengths, can be introduced.

Another method for introducing mutations into alpha-amylase-encoding DNA sequences is described in Nelson and Long, Analytical Biochemistry 180, 1989, pp. 147-151. It involves the 3-step generation of a PCR fragment, containing the desired mutation introduced by using a chemically synthesized DNA strand as one of the primers in the PCR reactions. From the PCR-generated fragment, a DNA fragment carrying the mutation may be isolated by cleavage with restriction endonucleases and reinserted into an expression plasmid.

### Random Mutagenesis

Random mutagenesis is suitably performed either as localised or region-specific random mutagenesis in at least three parts of the gene translating to the amino acid sequence shown in question, or within the whole gene.

The random mutagenesis of a DNA sequence encoding a parent alpha-amylase may be conveniently performed by use of any method known in the art.

In relation to the above, a further aspect of the present invenbon relates to a method for generating a variant of a parent alpha-amylase, e.g. wherein the variant exhibits altered or increased thermal stability relative to the parent, the method comprising:

(a) subjecting a DNA sequence encoding the parent alpha-amylase to random mutagenesis,

(b) expressing the mutated DNA sequence obtained in step (a) in a host cell, and

(c) screening for host cells expressing an alpha-amylase variant which has an altered property (e.g., pH-stability) relative to the parent alpha-amylase.

Step (a) of the above method of the invention is preferably performed using doped primers.

For instance, the random mutagenesis may be performed by use of a suitable physical or chemical mutagenizing agent, by use of a suitable oligonucleotide, or by subjecting the DNA sequence to PCR generated mutagenesis. Furthermore, the random mutagenesis may be performed by use of any combination of these mutagenizing agents. The mutagenizing agent may, e.g., be one that induces transitions, transversions, inversions, scrambling, deletions, and/or insertions.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues. When such agents are used, the mutagenesis is typically performed by incubating the DNA sequence encoding the parent enzyme to be mutagenized in the presence of the mutagenizing agent of choice under suitable conditions for the mutagenesis to take place, and selecting for mutated DNA having the desired properties.

When the mutagenesis is performed by the use of an oligonucleotide, the oligonucleotide may be doped or spiked with the three non-parent nucleotides during the synthesis of the oligonucleotide at the positions, which are to be changed. The doping or spiking may be done so that codons for unwanted amino acids are avoided. The doped or spiked oligonucleotide can be incorporated into the DNA encoding the alpha-amylase enzyme by any published technique, using e.g. PCR, LCR or any DNA polymerase and ligase as deemed appropriate.

Preferably, the doping is carried out using “constant random doping”, in which the percentage of wild-type and mutation in each position is predefined. Furthermore, the doping may be directed toward a preference for the introduction of certain nucleotides, and thereby a preference for the introduction of one or more specific amino acid residues. The doping may be made, e.g., so as to allow for the introduction of 90% wild type and 10% mutations in each position. An additional consideration in the choice of a doping scheme is based on genetic as well as protein-structural constraints. The doping scheme may be made using the DOPE program (see “Material and Methods” section), which, inter alia, ensures that introduction of stop codons is avoided.

When PCR-generated mutagenesis is used, either a chemically treated or non-treated gene encoding a parent alpha-amylase is subjected to PCR under conditions that increase the mis-incorporation of nucleotides (Deshler 1992; Leung et al., Technique, Vol.1, 1989, pp. 11-15).

A mutator strain of E. coil (Fowler et al., Molec. Gen. Genet., 133, 1974, pp. 179-191), S. cereviseae or any other microbial organism may be used for the random mutagenesis of the DNA encoding the alpha-amylase by, e.g., transforming a plasmid containing the parent glycosylase into the mutator strain, growing the mutator strain with the plasmid and isolating the mutated plasmid from the mutator strain. The mutated plasmid may be subsequently transformed into the expression organism.

The DNA sequence to be mutagenized may be conveniently present in a genomic or cDNA library prepared from an organism expressing the parent alpha-amylase. Alternatively, the DNA sequence may be present on a suitable vector such as a plasmid or a bacteriophage, which as such may be incubated with or otherwise exposed to the mutagenising agent. The DNA to be mutagenized may also be present in a host cell either by being integrated in the genome of said cell or by being present on a vector harboured in the cell. Finally, the DNA to be mutagenized may be in isolated form. It will be understood that the DNA sequence to be subjected to random mutagenesis is preferably a cDNA or a genomic DNA sequence.

In some cases it may be convenient to amplify the mutated DNA sequence prior to performing the expression step b) or the screening step c). Such amplification may be performed in accordance with methods known in the art, the presently preferred method being PCR-generated amplification using oligonucleotide primers prepared on the basis of the DNA or amino acid sequence of the parent enzyme.

Subsequent to the incubation with or exposure to the mutagenising agent, the mutated DNA is expressed by culturing a suitable host cell carrying the DNA sequence under conditions allowing expression to take place. The host cell used for this purpose may be one which has been transformed with the mutated DNA sequence, optionally present on a vector, or one which was carried the DNA sequence encoding the parent enzyme during the mutagenesis treatment. Examples of suitable host cells are the following: gram positive bacteria such as Bacillus subtilis, Bacillus licheniformis, Bacillus lentus, Bacillus brevis Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans or Streptomyces murinus, and gram-negative bacteria such as E. coli or Pseudomonas.

The mutated DNA sequence may further comprise a DNA sequence encoding functions permitting expression of the mutated DNA sequence.

### Localized Random Mutagenesis

The random mutagenesis may be advantageously localized to a part of the parent alpha-amylase in question. This may, e.g., be advantageous when certain regions of the enzyme have been identified to be of particular importance for a given property of the enzyme, and when modified are expected to result in a variant having improved properties. Such regions may normally be identified when the tertiary structure of the parent enzyme has been elucidated and related to the function of the enzyme.

The localized, or region-specific, random mutagenesis is conveniently performed by use of PCR generated mutagenesis techniques as described above or any other suitable technique known in the art. Alternatively, the DNA sequence encoding the part of the DNA sequence to be modified may be isolated, e.g., by insertion into a suitable vector, and said part may be subsequently subjected to mutagenesis by use of any of the mutagenesis methods discussed above.

### Alternative Methods of Providing Alpha-Amylase Variants

Alternative methods for providing variants of the invention include gene-shuffling method known in the art including the methods, e.g., described in WO 95/22625 (from Affymax Technologies N.V.) and WO 96/00343 (from Novo Nordisk ANS).

### Expression of Alpha-Amylase Variants Expresion Vectors

According to the invention, a DNA sequence encoding the variant produced by methods described above, or by any alternative methods known in the art, can be expressed, in enzyme form, using an expression vector which typically includes control sequences encoding a promoter, operator, ribosome binding site, translation initiation signal, and, optionally, a repressor gene or various activator genes.

The recombinant expression vector carrying the DNA sequence encoding an alpha-amylase variant of the invention may be any vector, which may conveniently be subjected to recombinant DNA procedures, and the choice of vector will often depend on the host cell into which it is to be introduced. Thus, the vector may be an autonomously replicating vector, i.e., a vector which exists as an extrachromosomal entity, the replication of which is independent of chromosomal replication, e.g., a plasmid, a bacteriophage or an extrachromosomal element, minichromosome or an artificial chromosome. Alternatively, the vector may be one which, when introduced into a host cell, is integrated into the host cell genome and replicated together with the chromosome(s) into which it has been integrated.

### Promoters

In the vector, the DNA sequence should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing th transcription of the DNA sequence encoding an alpha-amylase variant of the invention, especially in a bacterial host, are the promoter of the lac operon of E.coli, the Streptomyces coelicolor agarase gene dagA promoters, the promoters of the Bacillus licheniformis alpha-amylase gene (amyL), the promoters of the Bacillus stearothermophilus maltogenic amylase gene (amyM), the promoters of the Bacillus amyloliquefaciens alpha-amylase (amyQ), the promoters of the Bacillus subtilis xylA and xylB genes etc. For transcription in a fungal host, examples of useful promoters are those derived from the gene encoding A. oryzae TAKA amylase, Rhizomucor miehei aspartic proteinase, A. niger neutral alpha-amylase, A. niger acid stable alpha-amylase, A. niger glucoamylase, Rhizomucor miehei lipase, A. oryzae alkaline protease, A. oryzae triose phosphate isomerase or A. nidulans acetamidase.

### Transcription Terminators

The expression vector of the invention may also comprise a suitable transcription terminator and, in eukaryotes, polyadenylation sequences operably connected to the DNA sequence encoding the alpha-amylase variant of the invention. Termination and polyadenylation sequences may suitably be derived from the same sources as the promoter.

### Replication Sequences

The vector may further comprise a DNA sequence enabling the vector to replicate in the host cell in question. Examples of such sequences are the origins of replication of plasmids pUC19, pACYC177, pUB110, pE194, pAMB1 and pIJ702.

### Selectable Markers

The vector may also comprise a selectable marker, e.g., a gene the product of which complements a defect in the host cell, such as the dal genes from B. subtilis or B. licheniformis, or one which confers antibiotic resistance such as ampicillin, kanamycin, chloramphenicol or tetracyclin resistance. Furthermore, the vector may comprise Aspergillus selection markers such as amdS, argB, niaD and sC, a marker giving rise to hygromycin resistance, or the selection may be accomplished by co-transformation, e.g., as described in WO 91/17243.

### Secretion Sequences

While intracellular expression may be advantageous in some respects, e.g., when using certain bacteria as host cells, it is generally preferred that the expression is extracellular. In general, the Bacillus alpha-amylases mentioned herein comprise a preregion permitting secretion of the expressed protease into the culture medium. If desirable, this preregion may be replaced by a different preregion or signal sequence, conveniently accomplished by substitution of the DNA sequences encoding the respective preregions.

### Host Cells

The procedures used to ligate the DNA construct of the invention encoding an alpha-amylase variant, the promoter, terminator and other elements, respectively, and to insert them into suitable vectors containing the information necessary for replication, are well known to persons skilled in the art (cf., for instance, Sambrook et al., Molecular Cloning: A Laboratory Manual, 2nd Ed., Cold Spring Harbor, 1989).

The cell of the invention, either comprising a DNA construct or an expression vector of the invention as defined above, is advantageously used as a host cell in the recombinant production of an alpha-amylase variant of the invention. The cell may be transformed with the DNA construct of the invention encoding the variant, conveniently by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

The cell of the invention may be a cell of a higher organism such as a mammal or an insect, but is preferably a microbial cell, e.g., a bacterial or a fungal (including yeast) cell.

Examples of suitable bacteria are Gram-positive bacteria such as Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, or Streptomyces lividans or Streptomyces murinus, or gramnegative bacteria such as E.coli or pseudomonas. The transformation of the bacteria may, for instance, be effected by protoplast transformation or by using competent cells in a manner known per se.

The yeast organism may favorably be selected from a species of Saccharomyces or Schizosaccharomyces, e.g. Saccharomyces cerevisiae. The filamentous fungus may advantageously belong to a species of Aspergillus, e.g., Aspergillus oryzae or Aspergillus niger. Fungal cells may be transformed by a process involving protoplast formation and transformation of the protoplasts followed by regeneration of the cell wall in a manner known per se. A suitable procedure for transformation of Aspergillus host cells is described in EP 238 023.

### Method of Producing an Alpha-Amylase Variant of the Invention

In a yet further aspect, the present invention relates to a method of producing an alpha-amylase variant of the invention, which method comprises cultivating a host cell as described above under conditions conducive to the production of the variant and recovering the variant from the cells and/or culture medium.

The medium used to cultivate the cells may be any conventional medium suitable for growing the host cell in question and obtaining expression of the alpha-amylase variant of the invention. Suitable media are available from commercial suppliers or may be prepared according to published recipes (e.g., as described in catalogues of the American Type Culture Collection).

The alpha-amylase variant secreted from the host cells may conveniently be recovered from the culture medium by well-known procedures, including separating the cells from the medium by centrifugation or filtration, and precipitating proteinaceous components of the medium by means of a salt such as ammonium sulphate, followed by the use of chromatographic procedures such as ion exchange chromatography, affinity chromatography, or the like.

### Industrial Applications

Owing to their activity at alkaline pH values, the alpha-amylase variants of the invention are well suited for use in a variety of industrial processes, in particular the enzyme finds potential applications as a component in detergents, e.g., laundry, dishwashing and hard surface cleaning detergent compositions, but it may also be useful for desizing of textiles, fabrics and garments, beer making or brewing, in pulp and paper production, and further in the production of sweeteners and ethanol (see for instance U.S. Pat. No. 5,231,017—hereby incorporated by reference), such as fuel, drinking and industrial ethanol, from starch or whole grains.

### Starch Conversion

Conventional starch-conversion processes, such as liquefaction and saccharification processes are described, e.g., in U.S. Pat. No. 3,912,590 and EP patent publications Nos. 252,730 and 63,909, hereby incorporated by reference.

A “traditional” starch conversion process degrading starch to lower molecular weight carbohydrate components such as sugars or fat replacers includes a debranching step.

### Starch to Sugar Conversion

In the case of converting starch into a sugar the starch is depolymerized. A such depolymerization process consists of a Pre-treatment step and two or three consecutive process steps, viz. a liquefaction process, a saccharification process and dependent on the desired end product optionally an isomerization process.

### Pre-Treatment of Native Starch

Native starch consists of microscopic granules, which are insoluble in water at room temperature. When an aqueous starch slurry is heated, the granules swell and eventually burst, dispersing the starch molecules into the solution. During this “gelatinization” process there is a dramatic increase in viscosity. As the solids level is 30-40% in a typically industrial process, the starch has to be thinned or “liquefied” so that it can be handled. This reduction in viscosity is today mostly obtained by enzymatic degradation.

### Liquefaction

During the liquefaction step, the long chained starch is degraded into branched and linear shorter units (maltodextrins) by an alpha-amylase. The liquefaction process is carried out at 105-110° C. for 5 to 10 minutes followed by 1-2 hours at 95° C. The pH lies between 5.5 and 6.2. In order to ensure optimal enzyme stability under these conditions, 1 mM of calcium is added (40 ppm free calcium ions). After this treatment the liquefied starch will have a “dextrose equivalent” (DE) of 10-15.

### Saccharification

After the liquefaction process the maltodextrins are converted into dextrose by addition of a glucoamylase (e.g., AMG™) and a debranching enzyme, such as an isoamylase (U.S. Pat. No. 4,335,208) or a pullulanase (e.g., Promozyme™) (U.S. Pat. No. 4,560,651). Before this step the pH is reduced to a value below 4.5, maintaining the high temperature (above 95° C.) to inactivate the liquefying alpha-amylase to reduce the formation of short oligosaccharide called “panose precursors” which cannot be hydrolyzed properly by the debranching enzyme.

The temperature is lowered to 60° C., and glucoamylase and debranching enzyme are added. The saccharification process proceeds for 24-72 hours.

Normally, wh n denaturing the α-amylase after the liquefaction step about 0.2-0.5% of the saccharification product is the branched trisaccharide 6²-alpha-glucosyl maltose (panose) which cannot be degraded by a pullulanase. If active amylase from the liquefaction step is present during saccharification (i.e., no denaturing), this level can be as high as 1-2%, which is highly undesirable as it lowers the saccharification yield significantly.

### Isomerization

When the desired final sugar product is, e.g., high fructose syrup the dextrose syrup may be converted into fructose. After the saccharification process the pH is increased to a value in the range of 6-8, preferably pH 7.5, and the calcium is removed by ion exchange. The dextrose syrup is then converted into high fructose syrup using, e.g., an immmobilized glucoseisomerase (such as Sweetzyme™ IT).

### Ethanol Production

In general alcohol production (ethanol) from whole grain can be separated into 4 main steps

Milling

Liquefaction

Saccharification

Fermentation

### Milling

The grain is milled in order to open up the structure and allowing for further processing. Two processes are used wet or dry milling. In dry milling the whole kernel is milled and used in the remaining part of the process. Wet milling gives a very good separation of germ and meal (starch granules and protein) and is with a few exceptions applied at locations where there is a parallel production of syrups.

### Liquefaction

In the liquefaction process the starch granules are solubilized by hydrolysis to maltodextrins mostly of a DP higher than 4. The hydrolysis may be carried out by acid treatment or enzymatically by alpha-amylase. Acid hydrolysis is used on a limited basis. The raw material can be milled whole grain or a side stream from starch processing.

Enzymatic liquefaction is typically carried out as a three-step hot slurry process. The slurry is heated to between 60-95□C, preferably 80-85□C, and the enzyme(s) is (are) added. Then the slurry is jet-cooked at between 95-140□C, preferably 105-125□C, cooled to 60-95□C and more enzyme(s) is (are) added to obtain the final hydrolysis. The liquefaction process is carried out at pH 4.5-6.5, typically at a pH between 5 and 6. Milled and liquefied grain is also known as mash.

### Saccharification

To produce low molecular sugars DP₁₋₃ that can be metabolized by yeast, the maltodextrin from the liquefaction must be further hydrolyzed. The hydrolysis is typically done enzymatically by glucoamylases, alternatively alpha-glucosidases or acid alpha-amylases can be used. A full saccharification step may last up to 72 hours, however, it is common only to do a pre-saccharification of typically 40-90 minutes and then complete saccharification during fermentation (SSF). Saccharification is typically carried out at temperatures from 30-65□C, typically around 60□C, and at pH 4.5.

### Fermentation

Yeast typically from Saccharomyces spp. is added to the mash and the fermentation is ongoing for 24-96 hours, such as typically 35-60 hours. The temperature is between 26-34□C, typically at about 32□C, and the pH is from pH 3-6, preferably around pH 45.

Note that the most widely used process is a simultaneous saccharification and fermentation (SSF) process where there is no holding stage for the saccharification, meaning that yeast and enzyme is added together. When doing SSF it is common to introduce a pre-saccharification step at a temperature above 50□C, just prior to the fermentation.

### Distillation

Following the fermentation the mash is distilled to extract the ethanol.

The ethanol obtained according to the process of the invention may be used as, e.g., fuel ethanol; drinking ethanol, i.e., potable neutral spirits; or industrial ethanol.

### By-Products

Left over from the fermentation is the grain, which is typically used for animal feed either in liquid form or dried.

Further details on how to carry out liquefaction, saccharification, fermentation, distillation, and recovering of ethanol are well known to the skilled person.

According to the process of the invention the saccharification and fermentation may be carried out simultaneously or separately.

### Pulp and Paper Production

The alkaline alpha-amylase of the invention may also be used in the production of lignocellulosic materials, such as pulp, paper and cardboard, from starch reinforced waste paper and cardboard, especially where re-pulping occurs at pH above 7 and where amylases facilitate the disintegration of the waste material through degradation of the reinforcing starch. The alpha-amylase of the invention is especially useful in a process for producing a papermaking pulp from starch-coated printed-paper. The process may be performed as described in WO 95/14807, comprising the following steps:

a) disintegrating the paper to produce a pulp,

b) treating with a starch-degrading enzyme before, during or after step a), and

c) separating ink particles from the pulp after steps a) and b).

The alpha-amylases of the invention may also be very useful in modifying starch where enzymatically modified starch is used in papermaking together with alkaline fillers such as calcium carbonate, kaolin and clays. With the alkaline alpha-amylases of the invention it becomes possible to modify the starch in the presence of the filler thus allowing for a simpler integrated process.

### Desizing of Textiles, Fabrics and Garments

An alpha-amylase of the invention may also be very useful in textile, fabric or garment desizing. In the textile processing industry, alpha-amylases are traditionally used as auxiliaries in the desizing process to facilitate the removal of starch-containing size, which has served as a protective coating on weft yarns during weaving. Complete removal of the size coating after weaving is important to ensure optimum results in the subsequent processes, in which the fabric is scoured, bleached and dyed. Enzymatic starch breakdown is preferred because it does not involve any harmful effect on the fiber material. In order to reduce processing cost and increase mill throughput, the desizing processing is sometimes combined with the scouring and bleaching steps. In such cases, non-enzymatic auxiliaries such as alkali or oxidation agents are typically used to break down the starch, because traditional alpha-amylases are not very compatible with high pH levels and bleaching agents. The non-enzymatic breakdown of the starch size does lead to some fiber damage because of the rather aggressive chemicals used. Accordingly, it would be desirable to use the alpha-amylases of the invention as they have an improved performance in alkaline solutions. The alpha-amylases may be used alone or in combination with a cellulase when desizing cellulose-containing fabric or textile. Desizing and bleaching processes are well known in the art. For instance, such processes are described in WO 95/21247, U.S. Pat. No. 4,643,736, EP 119,920 hereby in corporate by reference.

Commercially available products for desizing include Aquazyme® and Aquazyme® Ultra from Novo Nordisk A/S.

### Beer Making

The alpha-amylases of the invention may also be very useful in a beer-making process; the alpha-amylases will typically be added during the mashing process.

### Detergent Compositions

The alpha-amylase of the invention may be added to and thus become a component of a detergent composition.

The detergent composition of the invention may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the invention provides a detergent additive comprising the enzyme of the invention. The detergent additive as well as the detergent composition may comprise one or more other enzymes such as a protease, a lipase, a cutinase, an amylase, a carbohydrase, a cellulase, a pectinase, a mannanase, an arabinase, a galactanase, a xylanase, an oxidase, e.g., a laccase, a pectate lyase, and/or a peroxidase.

In general the properties of the chosen enzyme(s) should be compatible with the selected detergent, (i.e., pH-optimum, compatibility with other enzymatic and nonenzymatic ingredients, etc.), and the enzyme(s) should be present in effective amounts.

Proteases: Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtlisin Carlsberg, subtilisin 309, subtilisin 147 and subtilisin 168 (described in WO 89/06279). Examples of trypsin-like pro-teases are trypsin (e.g., of porcine or bovine origin) and the Fusarium protease described in WO 89106270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include Alcalase®, Savinase®, Primase®, Duralase®, Esperase®, and Kannase® (Novo Nordisk A/S), Maxatase®, Maxacal, Maxapem®, Properase®, Purafect®, Purafect Oxp®, FN2®, and FN3® (Genencor International Inc.).

Lipases: Suitable lipases include those of bacterial or fungal origin. Chemically modified or protein engineered mutants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include Lipolasem and Lipolase Ultra™ (Novo Nordisk A/S).

Amylases: Suitable amylases (alpha and/or beta) include those of bacterial or fungal origin. Chemically modified or protein engineered mutants are included. Amylases include, for example, alpha-amylases obtained from Bacillus, e.g., a special strain of B. licheniformis, described in more detail in GB 1,296,839. Examples of useful alpha-amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Natalase™, Fungamyl™ and BAN™ (Novo Nordisk A/S), Rapidase™ and Purastar™ (from Genencor International Inc.).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellu-lases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novo Nordisk A/S), Clazinase®, and Puradax HA® (Genencor International Inc.), and KAC-500(B)® (Kao Corporation).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme® (Novo Nordisk A/S).

Pectate lyase: Many pectate lyases have been described in the art, see e.g. WO 99/27083 (Novozymes A/S) or WO 99/27084 (Novozymes A/S), both of which are incorporated herein by reference in their totality.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, e.g., granulate, a liquid, a slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonyl-phenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty adds. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric add according to established methods. Protected enzymes may be prepared according to the method disclosed in EP 238,216.

The detergent composition of the invention may be in any convenient form, e.g., a bar, a tablet, a powder, a granule, a paste or a liquid. A liquid detergent may be aqueous, typically containing up to 70% water and 0-30% organic solvent, or non-aqueous.

The detergent composition comprises one or more surfactants, which may be non-ionic including semi-polar and/or anionic and/or cationic and/or zwitterionic. The surfactants are typically present at a level of from 0.1% to 60% by weight.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

When included therein the detergent will usually contain from about 0.2% to about 40% of a non-ionic surfactant such as alcohol ethoxylate, nonyl-phenol ethoxylate, alkylpolyglycoside, alkyldimethylamine-oxide, ethoxylated fatty acid monoethanol-amide, fatty acid monoethanolamide, polyhydroxy alkyl fatty acid amide, or N-acyl N-alkyl derivatives of glucosamine (“glucamides”).

The detergent may contain 0-65% of a detergent builder or complexing agent such as zeolite, diphosphate, tripho-sphate, phosphonate, carbonate, citrate, nitrilotriacetic acid, ethylenediaminetetraacetic acid, diethylenetri-aminepen-taacetic acid, alkyl- or alkenylsuccinic acid, soluble silicates or layered silicates (e.g. SKS-6 from Hoechst).

The detergent may comprise one or more polymers. Examples are carboxymethylcellulose, poly(vinyl-pyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid co-polymers.

The detergent may contain a bleaching system, which may comprise a H₂O₂ source such as perborate or percarbonate which may be combined with a peracid-forming bleach activator such as tetraacetylethylenediamine or nonanoyloxyben-zenesul-fonate. Alternatively, the bleaching system may comprise peroxyacids of, e.g., the amide, imide, or sulfone type.

The enzyme(s) of the detergent composition of the inven-tion may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the com-position may be formulated as described in, e.g., WO 92/19709 and WO 92/19708.

The detergent may also contain other conventional detergent ingredients such as e.g. fabric conditioners including clays, foam boosters, suds suppressors, anti-corrosion agents, soil-suspending agents, anti-soil re-deposition agents, dy s, bactericides, optical brighteners, hydrotropes, tarnish inhibitors, or perfumes.

It is at present contemplated that in the detergent compositions any enzyme, in particular the enzyme of the invention, may be added in an amount corresponding to 0.01-100 mg of enzyme protein per liter of wash liquor, preferably 0.05-5 mg of enzyme protein per liter of wash liquor, in particular 0.1-1 mg of enzyme protein per liter of wash liquor.

The enzyme of the invention may additionally be incorporated in the detergent formulations disclosed in WO 97/07202, which is hereby incorporated as reference. Dishwash Deterget Compositions

The enzyme of the invention mat also be used in dish wash detergent compositions, including the following:

1) POWDER AUTOMATIC DISHWASHING COMPOSITION

| | |
|---|---|
| Nonionic surfactant | 0.4-2.5% |
| Sodium metasilicate | 0-20% |
| Sodium disilicate | 3-20% |
| Sodium triphosphate | 20-40% |
| Sodium carbonate | 0-20% |
| Sodium perborate | 2-9% |
| Tetraacetyl ethylene diamine (TAED) | 1-4% |
| Sodium sulphate | 5-33% |
| Enzymes | 0.0001-0.1% |

2) POWDER AUTOMATIC DISHWASHING COMPOSITION

| | |
|---|---|
| Nonionic surfactant | 1-2% |
| (e.g. alcohol ethoxylate) | |
| Sodium disilicate | 2-30% |
| Sodium carbonate | 10-50% |
| Sodium phosphonate | 0-5% |
| Trisodium citrate dihydrate | 9-30% |
| Nitrilotrisodium acetate (NTA) | 0-20% |
| Sodium perborate monohydrate | 5-10% |
| Tetraacetyl ethylene diamine (TAED) | 1-2% |
| Polyacrylate polymer | 6-25% |
| (e.g. maleic acid/acrylic acid copolymer) | |
| Enzymes | 0.0001-0.1% |
| Perfume | 0.1-0.5% |
| Water | 5-10 |

3) POWDER AUTOMATIC DISHWASHING COMPOSITION

| | |
|---|---|
| Nonionic surfactant | 0.5-2.0% |
| Sodium disilicate | 25-40% |
| Sodium citrate | 30-55% |
| Sodium carbonate | 0-29% |
| Sodium bicarbonate | 0-20% |
| Sodium perborate monohydrate | 0-15% |
| Tetraacetyl ethylene diamine (TAED) | 0-6% |
| Maleic acid/acrylic | 0-5% |
| acid copolymer | |
| Clay | 1-3% |
| Polyamino acids | 0-20% |
| Sodium polyacrylate | 0-8% |
| Enzymes | 0.0001-0.1% |

4) POWDER AUTOMATIC DISHWASHING COMPOSITION

| | |
|---|---|
| Nonionic surfactant | 1-2% |
| Zeolite MAP | 15-42% |
| Sodium disilicate | 30-34% |
| Sodium citrate | 0-12% |
| Sodium carbonate | 0-20% |
| Sodium perborate monohydrate | 7-15% |
| Tetraacetyl ethylene | 0-3% |
| diamine (TAED) | |
| Polymer | 0-4% |
| Maleic acid/acrylic acid copolymer | 0-5% |
| Organic phosphonate | 0-4% |
| Clay | 1-2% |
| Enzymes | 0.0001-0.1% |
| Sodium sulphate | Balance |

5) POWDER AUTOMATIC DISHWASHING COMPOSITION

| | |
|---|---|
| Nonionic surfactant | 1-7% |
| Sodium disilicate | 18-30% |
| Trisodium citrate | 10-24% |
| Sodium carbonate | 12-20% |
| Monopersulphate (2 KHSO₅.KHSO₄.K₂SO₄) | 15-21% |
| Bleach stabilizer | 0.1-2% |
| Maleic add/acrylic acid copolymer | 0-6% |
| Diethylene triamine pentaacetate, | 0-2.5% |
| pentasodium salt | |
| Enzymes | 0.0001-0.1% |
| Sodium sulphate, water | Balance |

6) POWDER AND LIQUID DISHWASHING COMPOSITION WITH
CLEANING SURFACTANT SYSTEM

| | |
|---|---|
| Nonionic surfactant | 0-1.5% |
| Octadecyl dimethylamine N-oxide dihydrate | 0-5% |
| 80:20 wt.C18/C16 blend of octadecyl dimethylamine | 0-4% |
| N-oxide dihydrate and hexadecyldimethyl amine N- | |
| oxide dihydrate | |
| 70:30 wt.C18/C16 blend of octadecyl bis | 0-5% |
| (hydroxyethyl)amine N-oxide anhydrous and | |
| hexadecyl bis | |
| (hydroxyethyl)amine N-oxide anhydrous | |
| C₁₃-C₁₅ alkyl ethoxysulfate with an average degree of | 0-10% |
| ethoxylation of 3 | |
| C₁₂-C₁₅ alkyl ethoxysulfate with an average degree of | 0-5% |
| ethoxylation of 3 | |
| C₁₃-C₁₅ ethoxylated alcohol with an average degree of | 0-5% |
| ethoxylation of 12 | |
| A blend of C₁₂-C₁₅ ethoxylated alcohols with an | 0-6.5% |
| average degree of ethoxylation of 9 | |
| A blend of C₁₃-C₁₅ ethoxylated alcohols with an | 0-4% |
| average degree of ethoxylation of 30 | |
| Sodium disilicate | 0-33% |
| Sodium tripolyphosphate | 0-46% |
| Sodium citrate | 0-28% |
| Citric acid | 0-29% |
| Sodium carbonate | 0-20% |
| Sodium perborate monohydrate | 0-11.5% |
| Tetraacetyl ethylene diamine (TAED) | 0-4% |
| Maleic add/acrylic acid copolymer | 0-7.5% |
| Sodium sulphate | 0-12.5% |
| Enzymes | 0.0001-0.1% |

7) NON-AQUEOUS LIQUID AUTOMATIC DISHWASHING
COMPOSITION

| | |
|---|---|
| Liquid nonionic surfactant (e.g. alcohol ethoxylates) | 2.0-10.0% |
| Alkali metal silicate | 3.0-15.0% |
| Alkali metal phosphate | 20.0-40.0% |
| Liquid carrier selected from higher | 25.0-45.0% |
| glycols, polyglycols, polyoxides, glycolethers | |
| Stabilizer (e.g. a partial ester of phosphoric acid and a | 0.5-7.0% |
| C₁₆-C₁₈ alkanol) | |
| Foam suppressor (e.g. silicone) | 0-1.5% |
| Enzymes | 0.0001-0.1% |

8) NON-AQUEOUS LIQUID DISHWASHING COMPOSITION

| | |
|---|---|
| Liquid nonionic surfactant (e.g. alcohol ethoxylates) | 2.0-10.0% |
| Sodium silicate | 3.0-15.0% |
| Alkali metal carbonate | 7.0-20.0% |
| Sodium citrate | 0.0-1.5% |
| Stabilizing system (e.g. mixtures of finely divided | 0.5-7.0% |
| silicone and low molecular weight dialkyl polyglycol | |
| ethers) | |
| Low molecule weight polyacrylate polymer | 5.0-15.0% |
| Clay gel thickener (e.g. bentonite) | 0.0-10.0% |
| Hydroxypropyl cellulose polymer | 0.0-0.6% |
| Enzymes | 0.0001-0.1% |
| Liquid carrier selected from higher lycols, polyglycols, | Balance |
| polyoxides and glycol ethers | |

9) THIXOTROPIC LIQUID AUTOMATIC DISHWASHING
COMPOSITION

| | |
|---|---|
| C₁₂-C₁₄ fatty acid | 0-0.5% |
| Block co-polymer surfactant | 1.5-15.0% |
| Sodium citrate | 0-12% |
| Sodium tripolyphosphate | 0-15% |
| Sodium carbonate | 0-8% |
| Aluminium tristearate | 0-0.1% |
| Sodium cumene sulphonate | 0-1.7% |
| Polyacrylate thickener | 1.32-2.5% |
| Sodium polyacrylate | 2.4-6.0% |
| Boric acid | 0-4.0% |
| Sodium formate | 0-0.45% |
| Calcium formate | 0-0.2% |
| Sodium n-decydiphenyl oxide disulphonate | 0-4.0% |
| Monoethanol amine (MEA) | 0-1.86% |
| Sodium hydroxide (50%) | 1.9-9.3% |
| 1,2-Propanediol | 0-9.4% |
| Enzymes | 0.0001-0.1% |
| Suds suppressor, dye, perfumes, water | Balance |

10) LIQUID AUTOMATIC DISHWASHING COMPOSITION

| | |
|---|---|
| Alcohol ethoxylate | 0-20% |
| Fatty acid ester sulphonate | 0-30% |
| Sodium dodecyl sulphate | 0-20% |
| Alkyl polyglycoside | 0-21% |
| Oleic acid | 0-10% |
| Sodium disilicate monohydrate | 18-33% |
| Sodium citrate dihydrate | 18-33% |
| Sodium stearate | 0-2.5% |
| Sodium perborate monohydrate | 0-13% |
| Tetraacetyl ethylene diamine (TAED) | 0-8% |
| Maleic add/acrylic acid copolymer | 4-8% |
| Enzymes | 0.0001-0.1% |

11) LIQUID AUTOMATIC DISHWASHING COMPOSITION
CONTAINING PROTECTED BLEACH PARTICLES

| | |
|---|---|
| Sodium silicate | 5-10% |
| Tetrapotassium pyrophosphate | 15-25% |
| Sodium triphosphate | 0-2% |
| Potassium carbonate | 4-8% |
| Protected bleach particles, e.g. chlorine | 5-10% |
| Polymeric thickener | 0.7-1.5% |
| Potassium hydroxide | 0-2% |
| Enzymes | 0.0001-0.1 % |
| Water | Balance |

11) Automatic dishwashing compositions as described in 1), 2), 3), 4), 6) and 10), wherein perborate is replaced by percarbonate.

12) Automatic dishwashing compositions as described in 1)-6) which additionally contain a manganese catalyst. The manganese catalyst may, e.g., be one of the compounds described in “Efficient manganese catalysts for low-temperature bleaching”, Nature 369, 1994, pp. 637-639.

### Uses

The present invention is also directed to methods for using an alpha-amylase variant of the invention in detergents, in particular laundry detergent compositions and dishwashing detergent compositions, hard surface cleaning compositions, and in composition for desizing of textiles, fabrics or garments, for production of pulp and paper, beer making, ethanol production, and starch conversion processes as described above.

The present invention is further described by the following examples, which should not be construed as limiting the scope of the invention.

### Materials & Methods

### Enzymes

KSM-K36: SEQ ID NO: 2, disclosed in EP 1,022,334 deposited as FERM BP 6945.

KSM-K38: SEQ ID NO: 4, disclosed in EP 1,022,334, deposited as FERM BP-6946.

Bacillus subtilis SHA273: Protease and amylase deleted Bacillus subtilis strain (disclosed in WO 95/10603).

### Detergent

Model detergent: A/P (Asia/Pacific) Model Detergent has the following composition: 20% STPP (sodium tripolyphosphate), 25% Na₂SO₄, 15% Na₂CO₃, 20% LAS (linear alkylbenzene sulfonate, Nansa 80S), 5% C₁₂-C₁₅ alcohol ethoxylate (Dobanol 25-7), 5% Na₂Si₂O₅, 0.3% NaCl.

Omo™ Multi Acao (Brazil),

Omo™ concentrated powder (EU) (Unilever)

Ariel Futur™ liquid (EU) (Procter and Gamble)

Commercial detergents containing alpha-amylase was inactivated by microwaves before wash.

### Plasmids

pTVB110 is a plasmid replicating in Bacillus subtilis by the use of origin of replication from pUB110 (Gryczan, T. J. (1978), J. Bact. 134:318-329). The plasmid further encodes the cat gene, conferring resistance towards chlorampenicol, obtained from plasmid pC194 (Horinouchi, S. and Weisblum, B. (1982), J. Bact. 150: 815-825). The plasmid harbors a truncated version of the Bacillus licheniformis alpha-amylase gene, amyL, such that the amyL promoter, signal sequence and transcription terminator are present, but the plasmid does not provide an amy-plus phenotype (halo formation on starch containing agar).

The alpha-amylase genes homologous to the KSM-K36 (SEQ ID NO: 1) and KSM-K38 (SEQ ID NO: 3) were cloned into the Pst1-Sal1 sites of pTVB110. The coding amylase gene was obtained by PCR reaction using purified genomic DNA from the Bacillus KSM-K36 strain as template and the DAX-8N (SEQ ID NO: 9) and DAX8C (SEQ ID NO: 10) primers.

### Methods:

### General Molecular Biology Methods

Unless otherwise mentioned the DNA manipulations and transformations were performed using standard methods of molecular biology (Sambrook et al. (1989); Ausubel et al. (1995); Harwood and Cutting (1990).

### Filter Screening Assay

The assay can be used to screening of alpha-amylase variants having an improved stability at high pH compared to the parent enzyme and alpha-amylase variants having an improved stability at high pH and medium temperatures compared to the parent enzyme depending of the screening temperature setting.

### High pH Filter Assay

Bacillus libraries are plated on a sandwich of cellulose acetate (OE 67, Schleicher & Schuell, Dassel, Germany)—and nitrocellulose filters (Protran-Ba 85, Schleicher & Schuell, Dassel, Germany) on TY agar plates with 10 micro g/ml kanamycin at 37° C. for at least 21 hours. The cellulose acetate layer is located on the TY agar plate.

Each filter sandwich is specifically marked with a needle after plating, but before incubation in order to be able to localize positive variants on the filter and the nitrocellulose filter with bound variants is transferred to a container with glycin-NaOH buffer, pH 8.6-10.6 and incubated at room temperature (can be altered from 10°-60° C.) for 15 min. The cellulose acetate filters with colonies are stored on the TY-plates at room temperature until use. After incubation, residual activity is detected on plates containing 1% agarose, 0.2% starch in glycin-NaOH buffer, pH 8.6-10.6. The assay plates with nitrocellulose filters are marked the same way as the filter sandwich and incubated for 2 hours. at room temperature. After removal of the filters the assay plates are stained with 10% Lugol solution. Starch degrading variants are detected as white spots on dark blue background and then identified on the storage plates. Positive variants are rescreened twice under the same conditions as the first screen.

### Low Calcium Filter Assay

The Bacillus library are plated on a sandwich of cellulose acetate (OE 67, Schleicher & Schuell, Dassel, Germany)—and nitrocellulose filters (Protran-Ba 85, Schleicher & Schuell, Dassel, Germany) on TY agar plates with a relevant antibiotic, e.g., kanamycin or chloramphenicol, at 37° C. for at least 21 hours. The cellulose acetate layer is located on the TY agar plate.

Each filter sandwich is specifically marked with a needle after plating, but before incubation in order to be able to localize positive variants on the filter and the nitrocellulose filter with bound variants is transferred to a container with carbonate/bicarbonate buffer pH 8.5-10 and with different EDTA concentrations (0.001 mM -100 mM). The filters are incubated at room temperature for 1 hour. The cellulose acetate filters with colonies are stored on the TY-plates at room temperature until use. After incubation, residual activity is detected on plates containing 1% agarose, 0.2% starch in carbonatelbicarbonate buffer pH 8.5-10. The assay plates with nitrocellulose filters are marked the same way as the filter sandwich and incubated for 2 hours at room temperature. After removal of the filters the assay plates are stained with 10% Lugol solution. Starch degrading variants are detected as white spots on dark blue background and then identified on the storage plates. Positive variants are rescreened twice under the same conditions as the first screen.

### Determination of Isoelectric Point

The pI is determined by isoelectric focusing (ex: Pharmacia, Ampholine, pH 3.5-9.3).

### Fermentation of Alpha-Amylases and Variants

Fermentation may be performed by methods well known in the art or as follows.

A B. subtilis strain harboring the relevant expression plasmid is streaked on a LB-agar plate with a relevant antibiotic, and grown overnight at 37° C. The colonies are transferred to 100 ml BPX media supplemented with a relevant antibiotic (for instance 10 mg/l chloroamphinicol) in a 500 ml shaking flask.

### Composition of BPX Medium

| | | |
|---|---|---|
| Potato starch | 100 | g/l |
| Barley flour | 50 | g/l |
| BAN 5000 SKB | 0.1 | g/l |
| Sodium caseinate | 10 | g/l |
| Soy Bean Meal | 20 | g/l |
| Na₂HPO₄, 12 H₂O | 9 | g/l |
| Pluronic ™ | 0.1 | g/l |

The culture is shaken at 37° C. at 270 rpm for 4 to 5 days.

Cells and cell debris are removed from the fermentation broth by centrifugation at 4500 rpm in 20-25 minutes. Afterwards the supernatant is filtered to obtain a completely clear solution. The filtrate is concentrated and washed on an UF-filter (10000 cut off membrane) and the buffer is changed to 20 mM Acetate pH 5.5. The UF-filtrate is applied on a S-sepharose F.F. and elution is carried out by step elution with 0.2 M NaCl in the same buffer. The eluate is dialysed against 10 mM Tris, pH 9.0 and applied on a Q-sepharose F.F. and eluted with a linear gradient from 0-0.3M NaCl over 6 column volumes. The fractions, which contain the activity (measured by the Phadebas assay) are pooled, pH was adjusted to pH 7.5 and remaining color was removed by a treatment with 0.5% W/vol. active coal in 5 minutes.

### Stability Determination

The amylase stability is measured using the method as follows:

The enzyme is incubated under the relevant conditions. Samples are taken at various time points, e.g., after 0, 5, 10, 15 and 30 minutes and diluted 25 times (same dilution for all taken samples) in assay buffer (0.1M 50 mM Britton buffer pH 7.3) and the activity is measured using the Phadebas assay (Pharmacia) under standard conditions pH 7.3, 37° C.

The activity measured before incubation (0 minutes) is used as reference (100%). The decline in percent is calculated as a function of the incubation time. The table shows the residual activity after, e.g., 30 minutes of incubation.

### Measurement of the Calcium- and pH-Dependent Stability

Normally industrial liquefaction processes runs using pH 6.0-6.2 as liquefaction pH and an addition of 40 ppm free calcium in order to improve the stability at 95° C.-105° C. Some of the herein proposed substitutions have been made in order to improve the stability at

1. lower pH than pH 6.2 and/or

2. at free calcium levels lower than 40 ppm free calcium.

Two different methods can be used to measure the alterations in stability obtained by the different substitutions in the alpha-amylase in question:

Method 1. One assay which measures the stability at reduced pH, pH 5.0, in the presence of 5 ppm free calcium.

10 micro g of the variant are incubated under the following conditions: A 0.1 M acetate solution, pH adjusted to pH 5.0, containing 5 ppm calcium and 5% w/w common corn starch (free of calcium). Incubation is made in a water bath at 95° C. for 30 minutes.

Method 2. One assay, which measure the stability in the absence of free calcium and where the pH is maintained at pH 6.0. This assay measures the decrease in calcium sensitivity:

10 micro g of the variant were incubated under the following conditions: A 0.1 M acetate solution, pH adjusted to pH 6.0, containing 5% w/w common corn starch (free of calcium). Incubation was made in a water bath at 95° C. for 30 minutes.

### Assays for Alpha-Amylase Activity

### 1. Phadebas Assay

Alpha-amylase activity is determined by a method employing Phadebas) tablets as substrate. Phadebas tablets (Phadebas® Amylase Test, supplied by Pharmacia Diagnostic) contain a cross-linked insoluble blue-colored starch polymer, which has been mixed with bovine serum albumin and a buffer substance and tabletted.

For every single measurement one tablet is suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH). The test is performed in a water bath at the temperature of interest. The alpha-amylase to be tested is diluted in x ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law). The dilution of the enzyme must therefore be adjusted to fit this criterion. Under a specified set of conditions (temp., pH, reaction time, buffer conditions) 1 mg of a given alpha-amylase will hydrolyze a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

### 2. Alternative Method

Alpha-amylase activity is determined by a method employing the PNP-G7 substrate. PNP-G7 which is a abbreviation for p-nitrophenyl-alpha,D-maltoheptaoside is a blocked oligosaccharide which can be cleaved by an endo-amylase. Following the cleavage, the alpha-Glucosidase included in the kit digest the substrate to liberate a free PNP molecule which has a yellow colour and thus can be measured by visible spectophometry at λ=405 nm. (400-420 nm.). Kits containing PNP-G7 substrate and alpha-Glucosidase is manufactured by Boehringer-Mannheim (cat. No. 1054635).

To prepare the substrate one bottle of substrate (BM 1442309) is added to 5 ml buffer (BM1442309). To prepare the alpha-glucosidase one bottle of alpha-Glucosidase (BM 1462309) is added to 45 ml buffer (BM1442309). The working solution is made by mixing 5 ml alpha-Glucosidase solution with 0.5 ml substrate.

The assay is performed by transforming 20 micro I enzyme solution to a 96 well microtitre plate and incubating at 25° C. 200 micro I working solution, 25° C. is added. The solution is mixed and pre-incubated 1 minute and absorption is measured every 15 secounds over 3 minutes at OD 405 nm.

The slope of the time dependent absorption-curve is directly proportional to the specific activity (activity per mg enzyme) of the alpha-amylase in question under the given set of conditions.

### Specific Activity Determination

The specific activity is determined as activity/mg enzyme using one of the methods described above. The manufactures instructions are followed (see also below under “Assay for alpha-amylase activity).

### Oxidation Stability Determination

Raw filtered culture broths with different vatiants of the invention are diluted to an amylase activity of 100 KNU/ml (defined above) in 50 mM of a Britton-Robinson buffer at pH 9.0 and incubated at 40° C. Subsequently H₂O₂ is added to a concentration of 200 mM, and the pH value is re-adjusted to 9.0. The activity is now measured after 15 seconds and after 5, 15, and 30 minutes. The absorbance of the resulting blue solution, measured spectrophotometrically at 620 nm, is a function of the alpha-amylase activity.

### Washing Performance

Washing performance is evaluated by washing soiled test swatches for 15 and 30 minutes at 25° C. and 40° C., respectively; at a pH in the range from 9-10.5; water hardness in the range from 6 to 15□dH; Ca:Mg ratio of from 2:1 to 4:1, in different detergent solutions (see above as described above in the Materials section) dosed from 3 to 5 g/l dependent on the detergent with the alpha-amylase variant in question.

The recombinant alpha-amylase variant is added to the detergent solutions at concentrations of for instance 0.01-5 mg/l. The test swatches aree soiled with orange rice starch (CS-28 swatches available from CFT, Center for Test Material, Holland).

After washing, the swatches are evaluated by measuring the remission at 460 nm using an Elrepho Remission Spectrophotometer. The results are expressed as ΔR=remission of the swatch washed with the alpha-amylase minus the remission of a swatch washed at the same conditions without the alpha-amylase.

### General Method for Random Mutagenesis by Use of the DOPE Program

The random mutagenesis may be carried out as follows:

1. Select regions of interest for modification in the parent enzyme

2. Decide on mutation sites and non-mutated sites in the selected region

3. Decide on which kind of mutations should be carried out, e.g. with respect to the desired stability and/or performance of the variant to be constructed

4. Select structurally reasonable mutations.

5. Adjust the residues selected by step 3 with regard to step 4.

6. Analyze by use of a suitable dope algorithm the nucleotide distribution.

7. If necessary, adjust the wanted residues to genetic code realism (e.g., taking into account constraints resulting from the genetic code (e.g. in order to avoid introduction of stop codons))(the skilled person will be aware that some codon combinations cannot be used in practice and will need to be adapted)

8. Make primers

9. Perform random mutagenesis by use of the primers

10. Select resulting α-amylase variants by screening for the desired improved properties.

Suitable dope algorithms for use in step 6 are well known in the art. One algorithm is described by Tomandl, D. et al., Journal of Computer-Aided Molecular Design, 11 (1997), pp. 29-38). Another algorithm, DOPE, is described in the following:

### The Dope Program

The “DOPE” program is a computer algorithm useful to optimize the nucleotide composition of a codon triplet in such a way that it encodes an amino acid distribution which resembles most the wanted amino acid distribution. In order to assess which of the possible distributions is the most similar to the wanted amino acid distribution, a scoring function is needed. In the “Dope” program the following function was found to be suited:

$$s ≡ {\prod }_{i = 1}^{N} {( \frac{{x}_{i}^{{y}_{i}}}{{y}_{i}^{{y}_{i}}} \frac{{( 1 - {x}_{i} )}^{1 - {y}_{i}}}{{( 1 - {y}_{i} )}^{1 - {y}_{i}}} )}^{{w}_{i}} ,$$

where the xᵢ's are the obtained amounts of amino acids and groups of amino acids as calculated by the program, yᵢ's are the wanted amounts of amino acids and groups of amino acids as defined by the user of the program (e.g. specify which of the 20 amino acids or stop codons are wanted to be introduced, e.g. with a certain percentage (e.g. 90% Ala, 3% Ile, 7% Val), and wᵢ's are assigned weight factors as defined by the user of the program (e.g., depending on the importance of having a specific amino acid residue inserted into the position in question). N is 21 plus the number of amino acid groups as defined by the user of the program. For purposes of this function 0° is defined as being 1.

A Monte-Carlo algorithm (one example being the one described by Valleau, J. P. & Whittington, S. G. (1977) A guide to Mont Carlo for statistical mechanics: 1 Highways. In “Stastistical Mechanics, Part A” Equlibrium Techniqeues ed. B. J. Berne, New York:

Plenum) is used for finding the maximum value of this function. In each iteration the following steps are performed:

1. A new random nucleotide composition is chosen for each base, where the absolute difference between the current and the new composition is smaller than or equal to d for each of the four nucleotides G,A,T,C in all three positions of the codon (see below for definition of d).

2. The scores of the new composition and the current composition are compared by the use of the function s as described above. If the new score is higher or equal to the score of the current composition, the new composition is kept and the current composition is changed to the new one. If the new score is smaller, the probability of keeping the new composition is exp(1000(new_score-current_score)).

A cycle normally consists of 1000 iterations as described above in which d is decreasing linearly from 1 to 0. One hundred or more cycles are performed in an optimization process. The nucleotide composition resulting in the highest score is finally presented.

### EXAMPLES

### Example 1

### Construction of Stabilised Amylase Variants

Stabilising amino acid substitutions can be introduced by the mega-primer-PCR method described by Sarkar and Sommer, 1990, BioTechniques 8: 404-407, using a mutagenesis primer and two specific primers binding upstreams and down-streams, respectively of both the point of mutation and the restriction sites to be used for cloning.

To introduce the substitutions: E84Q, N96D, A315S, A445V, G464N, N121D and N393H the following mutagenesis primers could be used:

| | |
|---|---|
| Primer Amrk752-E84Q: | |
| ctaaggcacagcttcaacgagctattgggtcc | (SEQ ID NO:11) |
| Primer Amrk752-N96D: | |
| ccttaaatctaatgatatcgatgtatacggagatg | (SEQ ID NO:12) |
| Primer Amrk752-A315S: | |
| ttataatttttaccggtcttcacaacaaggtgga | (SEQ ID NO:13) |
| Primer Amrk752-A445V: | |
| gtaggacgtcagaatgtaggacaaacatggac | (SEQ ID NO:14) |
| Primer Amrk752-G464N: | |
| ccgttacaattaataacgatggatggggcgaattc | (SEQ ID NO:15) |
| Primer Amrk23O-N121D: | |
| gcaagctgttcaagtagatccaacgaatcgttgg | (SEQ ID NO:16) |
| Primer Amrk230-N390H: | |
| gcttgatgcacgtcaagattacgcatatggcacg | (SEQ ID NO:17) |

—where the mutated codon is highlighted.

The amylase variant Amrk752 can be constructed by simultaneous introduction of the first five substitutions into SEQ 4 while Amrk230 can be constructed by introducing the last to substitutions into SEQ 4.

In a similar manner can Amrk299 be constructed on the basis of SEQ 4 by introducing the substitutions: T125S, S144P, I173L, D210E, N393H, V408I, R442Q, N444H, Q448A and G464S.

Wild type and variant amylases could be expressed in B.subtlis strains deficient of background amylase and protease activity and following be purified by conventional purifications methods.

### Example 2

### Activity at Alkaline pH

The relative activity of the amylases at alkaline pH was measured on culture broth, and the activity at pH 8 was defined to be 100% for comparison of the results in the table below. The Phadebas amylase assay system manufacted by Pharmacia AB was used in pH 10 buffer at 50° C. and with 15 min reaction time.

| Amylase | pH 8 | pH 10 |
|---|---|---|
| SEQ 4 | 100% | 6% |
| Amrk230 | 100% | 94% |
| Amrk299 | 100% | 19% |
| Amrk752 | 100% | 49% |

1. A variant of a parent alpha-amylase, comprising an alteration at one or more positions selected from the group of: 2,9,14,15,16,26,27,48,49,51,52,53,54,58,73,88,94,96,103,104,107,108,111,114,128,130, 133,138,140,142,144,148,149,156,161,165,166,168,171,173,174,178,179,180,181,183,184, 187,188,190,194,197,198,199,200,201,202,203,204,205,207,209,210,211,212,214,221,222, 224,228,230,233,234,237,239,241,242,252,253,254,255,260,264,265,267,275,276,277,280, 281,286,290,293,301,305,314,315,318,329,333,340,341,356,375,376,377,380,383,384,386, 389,399,403,404,405,406,427,441,444,453,454,472,479,480 wherein
(a) the alteration(s) are independently
(i) an insertion of an amino acid downstream of the amino acid which occupies the position,
(ii) a deletion of the amino acid which occupies the position, or
(iii) a substitution of the amino acid which occupies the position with a different amino add,
(b) the variant has alpha-amylase activity, and
(c) each position corresponds to a position of the amino acid sequence of the parent alpha-amylase having the amino acid sequence of the SM-36 alpaha-amylases shown in SEQ ID NO: 2:

2. The variant of claim 1, which variant has one or more of the following mutations:
G2P,A; M9I,L,F; H14Y; L15M,I,F,T; E16P; H26Y,Q,R,N; D27N,S,T; G48A,V,S,T; N49X; Q51X; A52X; D53E,Q,R; V54X; A58V,L,I,F; V73L,I,F; E84Q; G88X; D94X; N96Q; M103I,L,F; N104D; M/L107G,A,V,T,S,I,L,F; G108A; F111G,A,V,I,L,T; A114D,I,L,M,V,R; T125S; D128T,E; S130T,C; Y133F,H; W138F,Y; G140H,R,K,D,N; D142H,R,K,N; S144P; N148S; A149I; R156H,K,D,N; N161X; W165R; D166E; R168P; E171L,I,F; H173R,K,L; I173L; L174I,F; A178N,Q,R,K,H; N179G,A,T,S; T180N,Q,R,K,H; N181X; N183X; W184R,K; D187N,S,T; E188P,T,I,S; N190F; D194X; L197X; G198X; S199X; N200X; I201L,M,F,Y; D202X; F203L,I,F,M; S204X; H205X; E207Y,R; Q209V,L,I,F,M; E210X; E211Q; L212I,F; D214N,R,K,H; D221N; E222Q,T; D224N,Q; Y228F; L230I,F; I233A,V,L,F; K234N,Q; P237X; W239X; T241L,l,F,M; S242P,R; A252T; D253G,A,V,N; Q254K; D255N,Q,E,P; G260A; K264Q,S,T; D265N,Y; V267L,I,F,M; D275N,T; E276K; M277T,I,L,F; E280N,T,Q,S; M281H,I,L,F; V286X, preferably V286Y,L,I,F; Y290X; Y293H,F; S301G,A,D,K,E,R; R305A,K,Q,E,H,D,N; E314K,Q,R,S,T,H,N; A315K,R,S; 1318L,M,F; T329S; E333Q; A340R,K,N,D,Q,E; D341P,T,S,Q,N; G356Q,E,S,T,A; S375P; A376S; K377L,I,F,M; M380I,L,F; E383P,Q; L384I,F; D386N,Q,R,K,I,L; Q389K,R; Y399A,D,H; W403X; D404N; I405L,F; V406I,L,F,A,D; N427X; H441K,N,D,Q,E; R442Q; Q444E,K,R; A445V; Q448A; H453R,K,Q,N; A454S,T,P; G472R, N479Q,K,R; Q480K,R.

3. The variant of claims 1 or 2, wherein the variant has the following mutations:
N49I+L/M107A; N49L+L/M107A; G48A+N49I+L/M107A; G48A+N49L+L/M107; E188S,T,P+N190F+I201F+K264S; G48A+N49I+L/M107A+E188S,T,P+N190F+I201F+K264S; N190F+I201F; N190F+K264S; I201F+K264S; G140H+D142H+R156H,Y(+S144P); G140K+D142D+R156H,Y(+S144P); L197M+G198Y+S199A; L15T+E188S+Q209V+A376S+G472R; G48A+N49I+L/M107A+G140H+D142H+R156H,Y+E188P+N190F+I201F+K264S(+S144P); N49T+L/M107A+G140H+D142H+R156H,Y+E188P+N190F+I201F+K264S(+S144P);

4. The variant according to any of claims 1-3, wherein the parent alpha-amylase has an amino acid sequence which has a degree of identity to SEQ ID NO: 2 of at least 60%, preferably 70%, more preferably at least 80%, even more preferably at least about 90%, even more preferably at least 95%, even more preferably at least 97%, and even more preferably at least 99%.

5. The variant of any of claims 14, wherein the parent alpha-amylase is encoded by a nucleic acid sequence, which hydridizes under medium, preferred high stringency conditions, with the nucleic acid sequence of SEQ ID NO: 1 or 3.

6. The variant of claims 1-5, wherein the parent alpha-amylase is KSM-K38 shown in SEQ ID NO:4.

7. The variant of claims 1-6, which variant has altered pI, in particular a higher pI than the parent alpha-amylase.

8. A DNA construct comprising a DNA sequence encoding an alpha-amylase variant according to any one of claims 1 to 7.

9. A recombinant expression vector which carries a DNA construct according to claim 8.

10. A cell which is transformed with a DNA construct according to claim 8 or a vector according to claim 9.

11. A cell according to claim 10, which is a microorganism, preferably a bacterium or a fungus, in particular a gram-posifive bacterium, such as Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus or Bacillus thuringiensis.

12. A detergent additive comprising an alpha-amylase variant according to any one of claims 1-7, optionally in the form of a non-dusting granulate, stabilised liquid or protected enzyme.

13. A detergent additive according to claim 12, which contains 0.02-200 mg of enzyme protein/g of the additive.

14. A detergent additive according to claims 12 or 13, which additionally comprises another enzyme such as a protease, a lipase, a peroxidase, a pectate lyase, an amylase, or another amylolytic enzyme, such as maltogenic alpha-amylase or glucoamylase, mannanase, CGTase, and/or a cellulase.

15. A detergent composition comprising an alpha-amylase variant according to any of claims 1-7.

16. A detergent composition according to claim 15, which additionally comprises another enzyme such as a protease, a lipase, a peroxidase, a pectate lyase another amylolytc enzyme, glucoamylase, CGTase, mannanase, maltogenic amylase, and/or a cellulase.

17. A manual or automatic dishwashing detergent composition comprising an alpha-amylase variant according to any of claims 1-7.

18. A dishwashing detergent composition according to claim 17, which additionally comprises another enzyme such as a protease, a lipase, a peroxidase, a pectate lyase, an amylase, or another amylolytic enzyme, such as glucoamylase, CGTase, mannanase, maltogenic amylase and/or a cellulase.

19. A manual or automatic laundry washing composition comprising an alpha-amylase variant according to any of claims 1-7.

20. A laundry washing composition according to claim 19, which additionally comprises another enzyme such as a protease, a lipase, a peroxidase, a pectate lyase, an amylase, and/or another amylolytic enzyme, such as glucoamylase, CGTase, mannanase, maltogenic amylase and/or a cellulase.

21. Use of an alpha-amylase variant according to any one of claims 1-7 or a composition or an additive according to claims 12 to 20 for washing and/or dishwashing.

22. Use of an alpha-amylase variant according to any one of claims 1-7 or a composition or an additive according to claims 12 to 20 for textile desizing.

23. Use of an alpha-amylase variant according to any of claims 1-7 or a composition or an additive according to claims 12 to 20 for starch liquefaction.

24. Use,of an alpha-amylase variant according to any of claims 1-7 or a composition or an additive according to claims 12 to 20 for alcochol production, in particular ethanol production.
